(19) United States
(12) Patent Application Publication
(10) Pub. No.: US 2026/0263260 A1
(43) Pub. Date: Sep. 10, 2026

(54) FLUID COLLECTION ASSEMBLIES INCLUDING ONE OR MORE MOVEMENT ENHANCING FEATURES

(71) Applicant: PureWick Corporation, Covington, GA (US)

(72) Inventors: Dana Ahmad Abdelal, Marietta, GA (US); Nicolas Austerman, Atlanta, GA (US); Amanda Blackwell, Newborn, GA (US); Shaun Broussard, Conyers, GA (US); Jishen Cheng, Covington, GA (US); Rodrigo Fernandez, Loganville, GA (US); Eric Rehm, Greensboro, GA (US); Wendy Rodriguez, Covington, GA (US)

(21) Appl. No.: 19/554,125

(22) Filed: Mar. 2, 2026

Related U.S. Application Data
(63) Continuation of application No. 17/912,147, filed on Mar. 18, 2021, now Pat. No. 12,589,022.
(60) Provisional application No. 62/991,754, filed on Mar. 19, 2020.

(51) Int. Cl. A61F 5/44; A61F 5/451

(52) U.S. Cl. CPC A61F 5/4404; A61F 5/451

(57) ABSTRACT

An example fluid collection assembly includes a fluid impermeable barrier defining at least one opening, a chamber in fluid communication with the at least one opening, and at least one fluid outlet. The fluid collection assembly also includes at least one porous material disposed in the chamber. The fluid collection assembly also includes at least one conduit attached to the fluid outlet. The fluid collection assembly includes one or more movement enhancing features.

### CROSS-REFERENCE TO RELATED APPLICATIONS

This application is a continuation of U.S. application Ser. No. 17/912,147 filed on Sep. 16, 2022, which is a U.S. Nationalization of PCT International Application No. PCT/US2021/023001 filed on Mar. 18, 2021, which claims priority to U.S. Provisional Patent Application No. 62/991,754 filed on Mar. 19, 2020, the entire disclosure of each of which is incorporated herein, in its entirety, by this reference.

### BACKGROUND

A person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can be used to address some of these circumstances, such as incontinence. Unfortunately, urinary catheters can be uncomfortable, painful, and can lead to complications, such as infections. Additionally, bed pans, which are receptacles used for the toileting of bedridden patients are sometimes used. However, bedpans can be prone to discomfort, spills, and other hygiene issues.

### SUMMARY

Embodiments are directed to bodily fluids collection assemblies that include one or more movement enhancing features along with systems including and methods of using such bodily fluids collection assemblies. In an embodiment, a fluid collection assembly is disclosed. The fluid collection assembly includes a fluid impermeable barrier. The fluid impermeable barrier defines at least one opening, a chamber in fluid communication with the at least one opening, and at least one fluid outlet. The fluid collection assembly also includes at least one porous material disposed in the chamber and at least one conduit attached to the at least one fluid outlet. Further, the fluid collection assembly includes one or more movement enhancing features configured to allow increased movement by an individual using the fluid collection assembly without significantly increasing a likelihood that the fluid collection assembly leaks.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a top plan view of a fluid collection assembly that includes one or more movement enhancing features, according to an embodiment.

FIGS. 1B and 1C are cross-sectional views of the fluid collection assembly taken along line 1B-1B and 1C-1C, respectively, as shown in FIG. 1A.

FIG. 2A is a top plan view of a fluid collection assembly including a contact surface that is not flat, according to an embodiment.

FIG. 2B is a cross-sectional view of the fluid collection assembly taken along line 2B-2B, as shown in FIG. 2A.

FIG. 3 is a cross-sectional view of a fluid collection assembly that includes a protrusion that extends substantially completely across the opening thereof, according to an embodiment.

FIG. 4A is a top plan view of a fluid collection assembly that one or more grooves formed in the porous material that are movement enhancing features, according to an embodiment.

FIG. 4B is a cross-sectional view of the fluid collection assembly taken along line 4B-4B shown in FIG. 4B.

FIG. 5A is a top plan view of a fluid collection assembly that one or more perforations formed in the porous material that are movement enhancing features, according to an embodiment.

FIG. 5B is a cross-sectional view of the fluid collection assembly taken along line 5B-5B shown in FIG. 5B.

FIG. 6A is a top plan view of a fluid collection assembly that includes a fluid permeable element, according to an embodiment.

FIG. 6B is a cross-sectional view of the fluid collection assembly taken along line 6B-6B shown in FIG. 6A.

FIG. 7 is a top plan view of a fluid collection assembly that includes a fluid impermeable barrier having one or more movement enhancing features, according to an embodiment.

FIGS. 8 and 9 are isometric view of fluid collection assemblies exhibiting different shapes, according to different embodiments.

FIG. 10 is a cross-sectional view of a fluid collection assembly including a conduit extending from a back surface of the fluid impermeable barrier, according to an embodiment.

FIG. 11 is a top plan view of a plurality of fluid collection assemblies exhibiting different lengths, according to an embodiment.

FIG. 12 is a top plan view of a plurality of fluid collection assemblies exhibiting different widths, according to an embodiment.

FIG. 13 is a top plan view of a fluid collection assembly that is configured to have the conduit thereof secured to a location that is proximate to the fluid outlet thereof, according to an embodiment.

FIG. 14A is an isometric view of a fluid collection assembly, according to an embodiment.

FIG. 14B is a cross-sectional view of the fluid collection assembly of FIG. 14A taken along the plane 14B-14B of FIG. 14A.

FIG. 15 is a cross-sectional view of a portable vacuum source, according to an embodiment.

FIG. 16 is a block diagram of a system for fluid collection, according to an embodiment.

### DETAILED DESCRIPTION

Embodiments are directed to bodily fluids collection assemblies that include one or more movement enhancing features along with systems including and methods of using such bodily fluids collection assemblies. An example fluid collection assembly includes a fluid impermeable barrier defining at least one opening, a chamber in fluid communication with the at least one opening, and at least one fluid outlet. The fluid collection assembly also includes at least one porous material (e.g., at least one wicking material) disposed in the chamber. The fluid collection assembly also includes at least one conduit attached to the fluid outlet. During operation, the fluid collection assembly may receive one or more bodily fluids (e.g., urine) from an individual through the opening and into the chamber. The porous material may receive at least some of the bodily fluids that enter the chamber. A suction applied from the conduit to the chamber may direct the bodily fluids that chamber towards the conduit. The conduit may then remove the bodily fluids from the chamber.

The fluid collection assembly includes one or more movement enhancing features. The one or more movement enhancing features are configured to allow increased movement of an individual using the fluid collection assemblies disclosed herein compared to conventional fluid collection assemblies (i.e., substantially similar fluid collection assemblies that do not include any of the movement enhancing features disclosed herein). In an example, the fluid collection assemblies disclosed herein may press against and/or be positioned around the urethral opening of the individual thereby allowing the fluid collection assemblies to receive bodily fluids discharged from the urethral opening. With conventional fluid collection assemblies, movement by the individual (e.g., walking or changing the individual's position) may cause the fluid collection assembly to shift which, in turn, may cause one or more passageways (e.g., gaps) to form between the conventional fluid collection assemblies and the individual. Bodily fluids may leak (e.g., fail to enter the chamber or, when bodily fluids were previously received in the chamber, flow out of the chamber through the opening) through these passageways. As such, with conventional fluid collection assemblies, movement of the individual may be discouraged to prevent the bodily fluids from leaking. However, the movement enhancing features disclosed herein may be configured to allow increased movement of the individual without causing leaks. For example, at least some of the movement enhancing features may prevent the fluid collection assemblies disclosed herein from shifting when the individual moves. As such, at least some of the movement enhancing features disclosed herein may prevent the formation of passageways caused by the fluid collection assemblies shifting when the individual moves thereby minimizing the likelihood that the individual's movement cause leaks. In an example, the movement enhancing features may include a portable vacuum source and/or fluid storage container such that the individual using the fluid collection assemblies disclosed herein are not tethered to a non-portable vacuum source and/or fluid storage container.

It is noted that any of the fluid collection assemblies disclosed herein may include any one or any combination of the movement enhancing features disclosed herein, without limitation.

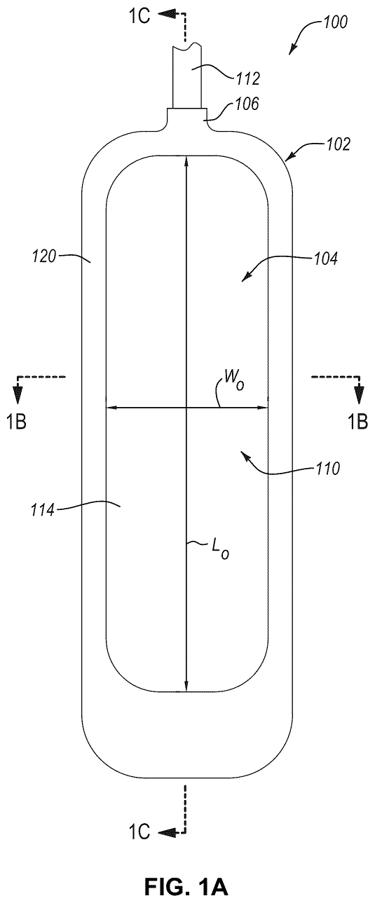

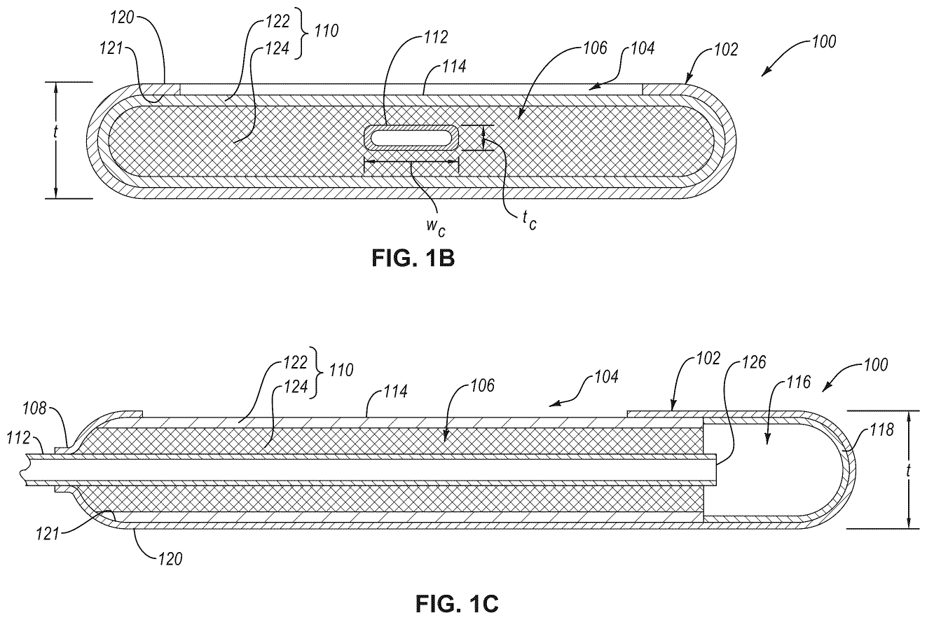

FIG. 1A is a top plan view of a fluid collection assembly 100 that includes one or more movement enhancing features, according to an embodiment. FIGS. 1B and 1C are cross-sectional views of the fluid collection assembly 100 taken along line 1B-1B and 1C-1C, respectively, as shown in FIG. 1A, according to an embodiment. The fluid collection assembly 100 includes a fluid impermeable barrier 102. The fluid impermeable barrier 102 defines at least one opening 104 configured to receive one or more bodily fluids from an individual. The fluid impermeable barrier 102 also defines a chamber 106 that is in fluid communication with the opening 104 and at least one fluid outlet 108. The fluid collection assembly 100 also include at least one porous material 110 disposed in the chamber 106 and at least one conduit 112 attached to the fluid outlet 108. The fluid collection assembly 100 also include one or more movement enhancing features.

The porous material 110 includes a contact surface 114. The contact surface 114 of the porous material 110 is the surface of the porous material 110 that extends across the opening 104 and, thus, is the surface of the porous material 110 that may contact the individual (e.g., contact or be positioned proximate to the urethral opening of the individual). In an embodiment, the contact surface 114 may be a movement enhancing feature of the fluid collection assembly 100 when the contact surface 114 is substantially flat, as shown in FIGS. 1B and 1C. For example, the vaginal region (collectively referred to as “vaginal region”) exhibits generally flat and/or convex shape. Some conventional fluid collection assemblies include a contact surface exhibiting a generally convex shape. The generally convex shape of the conventional fluid collection assemblies allow the contact surface thereof to press against labia folds of the individual and may even partially penetrate between the labia folds of the individual. However, lateral shifts in the conventional fluid collection assemblies (e.g., shifts in a direction perpendicular to a longitudinal length of the conventional fluid collection assemblies) caused by movement of the individual mitigates any benefit of the convex shape of the convex contact surface. In other words, the lateral shift of the conventional fluid collection assemblies caused by movement of the individual may cause leaks. In contrast, the substantially flat contact surface 114 of the porous material 110 of the fluid collection assembly 100 may continually press against the labia folds of the individual even when the fluid collection assembly laterally shifts. As such, the substantially flat contact surface 114 allows the individual to move without significantly increasing the risk of leaks. Further, the substantially flat contact surface 114 generally corresponds to the portions of the vaginal region that are substantially flat and is more easily bent into a concave shape that corresponds to the convex shape of the vaginal region than the conventional fluid collection assemblies. Causing the substantially flat contact surface 114 to corresponds to the shape of the vaginal region decreases the likelihood that the fluid collection assembly 100 shifts when the individual moves compared to the convention fluid collection assemblies.

The opening 104 may exhibit a maximum length L_{O} measured parallel to a longitudinal axis of the fluid collection assembly 100 and a maximum width W_{O} measured perpendicularly to the maximum length L_{O}. In an embodiment, the opening 104 may form a movement enhancing feature of the fluid collection assembly 100 when a ratio of the maximum width W_{O} relative to the maximum length L_{O} (i.e., W_{O}/L_{O}) is about 0.35 or greater, such as about 0.4 or greater, about 0.45 or greater, about 0.5 or greater, about 0.55 or greater, about 0.6 or greater, about 0.65 or greater, about 0.7 or greater, about 0.75 or greater, about 0.8 or greater, about 0.85 or greater, about 0.9 or greater, about 0.95 or greater, about 1.0 or greater, or in ranges of about 0.35 to about 0.45, about 0.4 to about 0.5, about 0.45 to about 0.55, about 0.5 to about 0.6, about 0.55 to about 0.65, about 0.6 to about 0.7, about 0.65 to about 0.75, about 0.7 to about 0.8, about 0.75 to about 0.85, about 0.8 to about 0.9, about 0.85 to about 0.95, or about 0.9 to about 1.0. For example, the increased width W_{O} of the opening 104 relative to the length L_{O} allows the fluid collection assembly 100 to accommodate larger lateral shifts caused by the individual moving without significantly increasing the likelihood of leaks compared to conventional fluid collection assemblies.

In an embodiment, the one or more movement enhancing features includes forming the fluid collection assembly 100 to exhibit a high degree of flexibility. The high degree of flexibility of the fluid collection assembly 100 allows the contact surface 114 to correspond to the shape of the vaginal region. As previously discussed, the fluid collection assembly 100 is less likely to shift when the fluid collection assembly 100 exhibits a shape that corresponds to the shape of the vaginal region. Further, movement of the individual may cause the shape of the vaginal region to change. For example, an individual moving from a sitting position to a laying or standing position may cause the vaginal region to straighten and movement of the thighs of the individual may cause the vaginal region to be compressed or pulled. However, the high flexibility of the fluid collection assembly 100 allows the contact surface 114 to better conform to the changes of the shape of the vaginal region thereby allowing greater movement of the individual without significantly increasing the likelihood that the fluid collection assembly 100 leaks.

In an embodiment, the fluid collection assembly 100 exhibits a high degree of flexibility because the fluid collection assembly 100 exhibits a relatively small maximum thickness t since the flexibility of the fluid collection assembly 100 is directly proportional to the thickness t thereof. For example, conventional fluid collection assemblies may exhibit a maximum thickness of about 25 mm or greater. However, the fluid collection assembly 100 may exhibit a maximum thickness that is about 25 mm or less which allows the fluid collection assembly 100 to exhibit a flexibility that is greater than the conventional fluid collection assemblies. The increased flexibility may allow the fluid collection assembly 100 to better conform to the vaginal region when the individual moves than the conventional fluid collection assemblies. In an example, the fluid collection assembly 100 may exhibit a maximum thickness t that is about 22.5 mm or less, about 20 mm or less, about 17.5 mm or less, about 15 mm or less, about 12.5 mm or less, about 10 mm or less, about 8 mm or less, about 6 mm or less, about 5 mm or less, about 4 mm or less, about 3 mm or less, about 2 mm or less, about 1 mm or less, or in ranges of about 1 mm to about 3 mm, about 2 mm to about 4 mm, about 3 mm to about 5 mm, about 4 mm to about 6 mm, about 5 mm to about 8 mm, about 6 mm to about 10 mm, about 8 mm to about 12.5 mm, about 10 mm to about 15 mm, about 12.5 mm to about 17.5 mm, about 15 mm to about 20 mm, about 17.5 mm to about 22.5 mm, or about 20 mm to about 25 mm.

The thickness t of the fluid collection assembly 100 may be selected based on the amount of movement that the individual is expected to make. In an example, the thickness t of the fluid collection assembly 100 may be about 10 mm to about 20 mm when the fluid collection assembly 100 is configured to allow limited movement of the individual, such as minor movement (e.g., adjusting the position of the thighs) while the individual remains in a constant position (e.g., remains laying down or remains sitting). In an example, the thickness t of the fluid collection assembly 100 may be about 5 mm to about 15 mm when the fluid collection assembly 100 is configured to allow the individual to change their position, such as allowing the individual to change between a laying position and a sitting position. In an example, the thickness t of the fluid collection assembly 100 may be about 1 mm to about 10 mm when the fluid collection assembly 100 is configured to be used while the individual walks.

The fluid collection assembly 100 exhibits a generally flat elongated shape that may allow the fluid collection assembly 100 to fit, at least partially, in the space and/or folds between the thighs, the perineum, and the vaginal region. The ability of the fluid collection assembly 100 to fit, at least partially, in the space and/or folds between the thighs, the perineum, and the vaginal region may allow the fluid collection assembly 100 exhibit the thickness t that is about 25 mm or less due to the relative shape of the fluid collection assembly 100. Additionally, the ability of the fluid collection assembly 100 to fit, at least partially, in the space and/or folds between the thighs, the perineum, and the vaginal region may allow the fluid collection assembly 100 to exhibit a maximum width (measured between opposing outer surfaces of the fluid impermeable barrier 102) that is about 25 mm or greater, unlike at least some conventional fluid collection assemblies which may exhibit a maximum width that is about 25 mm. For example, the fluid collection assembly 100 may exhibit a maximum width that is about 30 mm or greater, about 40 mm or greater, about 50 mm or greater, about 60 mm or greater, about 70 mm or greater, about 80 mm or greater, about 90 mm or greater, about 100 mm or greater, or in ranges of about 25 mm to about 40 mm, about 30 mm to about 50 mm, about 40 mm to about 60 mm, about 50 mm to about 70 mm, about 60 mm to about 80 mm, about 70 mm to about 90 mm, or about 80 mm to about 100 mm. The larger maximum width of the fluid collection assembly 100 allows the opening 104 to exhibit the relatively large width W_{O} relative to the length L_{O} of the opening 104.

The conduit 112 may exhibit a relatively flat cross-sectional shape due to the relatively small thickness t of the fluid collection assembly 100. For example, the conduit 112 may exhibit a conduit width Wc and a conduit thickness tc measured perpendicularly to the conduit width Wc. Due to the relatively flat cross-sectional shape of the conduit 112, the conduit width Wc may be greater than the conduit thickness tc thereby allowing the conduit 112 to be able to remove sufficient quantities of bodily fluids from the chamber 106. The smaller conduit thickness tc relative to the conduit width Wc may allow the conduit 112 to extend within the chamber 106 and behind the porous material 110 from the fluid outlet 108 to the fluid reservoir 116.

As previously discussed, the fluid collection assembly 100 may exhibits a highly flexibility. In an embodiment, the fluid collection assembly 100 exhibits the high flexibility thereof by decreasing the thickness of the fluid impermeable barrier 102 since the flexibility of the fluid collection assembly 100 depends, in part, on thickness of the fluid impermeable barrier 102. For example, at least some conventional fluid collection assemblies include a fluid impermeable barrier 102 exhibiting at thickness that is about 1 mm or greater. However, in some of the embodiments disclosed herein, the fluid impermeable barrier 102 may exhibit a thickness that is about 0.9 mm or less, about 0.8 mm or less, about 0.7 mm or less, about 0.6 mm or less, about 0.5 mm or less, about 0.4 mm or less, about 0.3 mm or less, about 0.2 mm or less, about 0.1 mm or less, or in ranges of about 0.05 mm to about 0.2 mm, about 0.1 mm to about 0.3 mm, about 0.2 mm to about 0.4 mm, about 0.3 mm to about 0.5 mm, about 0.4 mm to about 0.6 mm, about 0.5 mm to about 0.7 mm, about 0.6 mm to about 0.8 mm, about 0.7 mm to about 0.9 mm. The thickness of the fluid impermeable barrier 102 may depend on several factors. In an example, the thickness of the fluid impermeable barrier 102 may be selected based on the amount of movement that the fluid collection assembly 100 is configured to allow. For instance, the thickness of the fluid impermeable barrier 102 may be about 0.7 mm to about 0.9 mm when the fluid collection assembly 100 allows limited movement, about 0.3 mm to about 0.7 mm when the fluid collection assembly 100 allows the individual to changes between a standing, a sitting, and/or a lying position, or about 0.3 mm or less when the fluid collection assembly is configured to be used while the individual walks. In an example, the thickness of the fluid impermeable barrier 102 may be selected based on the maximum thickness of the fluid collection assembly 100.

Referring to FIG. 1C, the fluid collection assembly 100 may include a fluid reservoir 116 that is a substantially unoccupied portion of the chamber 106. The fluid reservoir 116 may be defined between the fluid impermeable barrier 102 and porous material 110. The bodily fluids that are in the chamber 106 may flow through the porous material 110 to the fluid reservoir 116 and the fluid reservoir 116 may retain of the bodily fluids therein. In an embodiment, when the fluid collection assembly 100 exhibits a high flexibility, the portion of the fluid impermeable barrier 102 defining the fluid reservoir 116 may collapse since the high flexibility of the fluid collection assembly 100 may not provide enough support to maintain the shape of the fluid impermeable barrier 102. Collapsing the fluid impermeable barrier 102 may decrease the volume of the bodily fluids that may be held in the fluid reservoir 116 and may cause the fluid impermeable barrier 102 to at least partially obstruct the conduit 112. As such, in an embodiment, the movement enhancing features of the fluid collection assembly 100 may include a fluid reservoir reinforcement structure 118. The fluid reservoir reinforcement structure 118 is configured to provide additional structure to the portions of the fluid impermeable barrier 102 that define the fluid reservoir 116, thereby preventing or inhibiting the fluid impermeable barrier 102 from collapsing.

In an embodiment, the fluid reservoir reinforcement structure 118 is formed by increasing the thickness of the portion of the fluid impermeable barrier 102 that defines the fluid reservoir 116 relative to the rest of the fluid impermeable barrier 102. Increasing the thickness of the fluid impermeable barrier 102 increases the rigidity of the fluid impermeable barrier 102 and may inhibit the fluid impermeable barrier 102 from collapsing. In an embodiment, as shown, the fluid reservoir reinforcement structure 118 is distinct from the fluid impermeable barrier 102. In such an embodiment, the fluid reservoir reinforcement structure 118 may be formed from the same material or one or more different materials that the portion of the fluid impermeable barrier 102 that defines the fluid reservoir 116. When the fluid reservoir reinforcement structure 118 is formed from the same material as the fluid impermeable barrier 102, the fluid reservoir reinforcement structure 118 effectively increases the thickness of the portion of the fluid impermeable barrier 102 that defines the fluid reservoir 116. When the fluid reservoir reinforcement structure 118 is formed from a material that is different than the fluid impermeable barrier 102, the fluid reservoir reinforcement structure 118 may be formed from a material exhibiting an elasticity (i.e., Young's modulus) that is greater than the fluid impermeable barrier 102. Examples of material that may exhibit an elasticity greater than the rest of the fluid impermeable barrier 102 includes epoxy, acetal, polyester, a metal (e.g., a metal foil), or any other suitable material. Since the fluid reservoir reinforcement structure 118 is formed from a different material than the fluid impermeable barrier 102, the fluid reservoir reinforcement structure 118 may be attached to the fluid impermeable barrier 102 using any suitable technique, such as with an adhesive, ultrasonic welding, heat staking, etc.

In an embodiment, when the fluid reservoir reinforcement structure 118 is distinct from the fluid impermeable barrier 102, the fluid reservoir reinforcement structure 118 may be disposed on (e.g., attached to) an outer surface 120 of the fluid impermeable barrier 102 or may be disposed on (e.g., attached to) an inner surface 121 of the fluid impermeable barrier 102.

As previously discussed, the fluid impermeable barrier 102 at least partially defines the opening 104 and the chamber 106 (e.g., interior region). For example, at least one inner surface 121 of the fluid impermeable barrier 102 at least partially defines the chamber 106 within the fluid collection assembly 100. The fluid impermeable barrier 102 temporarily stores the bodily fluids in the chamber 106. The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface 120 of the fluid impermeable barrier 102 may be formed from a soft and/or smooth material, thereby reducing chaffing. During use, a portion of the outer surface 120 of the fluid impermeable barrier 102 may contact the wearer. The fluid impermeable barrier 102 may be sized and shaped to fit in the gluteal cleft between the legs of an individual.

The opening 104 provides an ingress route for bodily fluids to enter the chamber 106. The opening 104 may be defined by the fluid impermeable barrier 102 such as by an inner edge of the fluid impermeable barrier 102. For example, the opening 104 is formed in and extends through the fluid impermeable barrier 102, from the outer surface 120 to the inner surface 121, thereby enabling the bodily fluids to enter the chamber 106 from outside of the fluid collection assembly 100. In an embodiment, the opening 104 may be an elongated hole (the length L_{O} is more than 50% greater than the width W_{O}) in the fluid impermeable barrier 102. For example, the opening 104 may be defined as a cut-out in the fluid impermeable barrier 102. The opening 104 may be located and shaped to be positioned adjacent to a female urethral opening.

The fluid collection assembly 100 may be positioned proximate to the female urethral opening and urine may enter the chamber 106 of the fluid collection assembly 100 via the opening 104. The fluid collection assembly 100 is configured to receive the bodily fluids into the chamber 106 via the opening 104. When in use, the opening 104 may extend from a first location above the urethral opening (e.g., at or near the top of the urethral opening or the pubic hair) to a second location below the urethral opening (e.g., at or near the anus or the vaginal opening).

In some examples, as previously discussed, the fluid impermeable barrier 102 may define an fluid outlet 108 sized to receive the conduit 112. The at least one conduit 112 may be disposed in the chamber 106 or otherwise in fluid communication with the chamber 106 via the fluid outlet 108. The fluid outlet 108 may be sized and shaped to form an at least substantially fluid tight seal against the conduit 112 or the at least one tube thereby substantially preventing the bodily fluids from escaping the chamber 106.

The fluid impermeable barrier 102 may include markings thereon (not shown), such as one or more markings to aid a user in aligning the fluid collection assembly 100 on the wearer. For example, a line on the fluid impermeable barrier 102 (e.g., opposite the opening 104) may allow a healthcare professional to align the opening 104 over the urethral opening of the individual wearing the fluid collection assembly 100. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes porous material 110 disposed in the chamber 106. The porous material 110 may cover at least a portion (e.g., all) of the opening 104. The porous material 110 is exposed to the environment outside of the chamber 106 through the opening 104. The permeable properties referred to herein may be wicking, capillary action, absorption, diffusion, or other similar properties or processes, and are referred to herein as “permeable” and/or “porous.” The porous material 110 may also wick the bodily fluids generally towards an interior of the chamber 106, as discussed in more detail below. The porous material 110 may include one or more of a fluid permeable membrane 122 or a fluid permeable support 124.

In an embodiment, at least a portion of the porous material 110 may be a wicking material configured to wick and/or allow transport any of the bodily fluids away from the opening 104, thereby preventing bodily fluids from escaping the chamber 106. The porous material 110 may not include absorption of the bodily fluids into at least a portion of the wicking material. Put another way, substantially no absorption or solubility of the bodily fluids into the porous material may take place after the porous material is exposed to the bodily fluids. While no absorption is desired, the term “substantially no absorption” may allow for nominal amounts of absorption and/or solubility of the bodily fluids into the porous material (e.g., absorbency), such as about 30 wt% of the dry weight of the wicking material, about 20%, about 10%, about 7 wt %, about 5 wt %, about 3 wt %, about 2 wt %, about 1 wt %, or about 0.5 wt % of the dry weight of the wicking material. In an embodiment, the porous material 110 may include at least one absorbent or adsorbent material.

The fluid collection assembly 100 may include the fluid permeable membrane 122 disposed in the chamber 106. The fluid permeable membrane 122 may cover at least a portion (e.g., all) of the opening 104. The fluid permeable membrane 122 may be composed to pull/push the bodily fluids away from the opening 104, thereby preventing the bodily fluids from escaping the chamber 106.

The fluid permeable membrane 122 may include any material that may be permeable to the bodily fluids. For example, the fluid permeable membrane 122 may include fabric, such as a gauze (e.g., a silk, linen, or cotton gauze), another soft fabric, another smooth fabric, or any of the other porous material disclosed herein. Forming the fluid permeable membrane 122 from gauze, soft fabric, and/or smooth fabric may reduce chaffing caused by the fluid collection assembly 100.

The fluid collection assembly 100 may include the fluid permeable support 124 disposed in the chamber 106. The fluid permeable support 124 is configured to support the fluid permeable membrane 122 since the fluid permeable membrane 122 may be formed from a relatively foldable, flimsy, or otherwise easily deformable material. For example, the fluid permeable support 124 may be positioned such that the fluid permeable membrane 122 is disposed between the fluid permeable support 124 and the fluid impermeable barrier 102. As such, the fluid permeable support 124 may support and maintain the position of the fluid permeable membrane 122. The fluid permeable support 124 may include any material that may be permeable to the bodily fluids, such as any of the fluid permeable membrane materials disclosed herein above. For example, the fluid permeable membrane material(s) may be utilized in a more dense or rigid form than in the fluid permeable membrane 122 when used as the fluid permeable support 124. The fluid permeable support 124 may be formed from any fluid permeable material that is less deformable than the fluid permeable membrane 122. For example, the fluid permeable support 124 may include a porous polymer (e.g., nylon, polyester, polyurethane, polyethylene, polypropylene, etc.) structure (e.g., spun fibers such as spun nylon fibers) or an open cell foam. In some examples, the fluid permeable support 124 may be formed from a natural material, such as cotton, wool, silk, or combinations thereof. In such examples, the material may have a coating to prevent or limit absorption of the bodily fluids into the material, such as a water repellent coating. In some examples, the fluid permeable support 124 may be formed from fabric, felt, gauze, or combinations thereof. In some examples, the fluid permeable membrane 122 may be optional. For example, the porous material 110 may include only the fluid permeable support 124. In some examples, the fluid permeable support 124 may be optionally omitted from the fluid collection assembly 100. For example, the porous material 110 may only include the fluid permeable membrane 122.

In an embodiment, the fluid permeable membrane 122 and the fluid permeable support 124 are wicking materials. In such an embodiment, the fluid permeable support 124 may have a greater ability to wick the bodily fluids than the fluid permeable membrane 122, such as to move the bodily fluids inwardly from the outer surface 120 of the fluid collection assembly 100. In some examples, the wicking ability of the fluid permeable support 124 and the fluid permeable membrane 122 may be substantially the same.

In an embodiment, not shown, the fluid permeable membrane 122 and the fluid permeable support 124 may at least substantially completely fill the portions of the chamber 106 that are not occupied by the conduit 112. In an embodiment, as previously discussed, the fluid permeable membrane 122 and the fluid permeable support 124 may not substantially completely fill the portions of the chamber 106 that are not occupied by the conduit 112. In such an example, the fluid collection assembly 100 includes the fluid reservoir 116 disposed in the chamber 106.

The fluid reservoir 116 is a substantially unoccupied portion of the chamber 106. The fluid reservoir 116 may be defined between the fluid impermeable barrier 102 and one or both of the fluid permeable membrane 122 and fluid permeable support 124. The bodily fluids that are in the chamber 106 may flow through the fluid permeable membrane 122 and/or fluid permeable support 124 to the fluid reservoir 116. The fluid reservoir 116 may retain of the bodily fluids therein. The bodily fluids that are in the chamber 106 may flow through the fluid permeable membrane 122 and/or fluid permeable support 124 and, optionally, to the fluid reservoir 116. The fluid impermeable barrier 102 may retain the bodily fluids in the fluid reservoir 116. The fluid reservoir 116 may be located in a portion of the chamber 106 that is designed to be located in a gravimetrically low point of the fluid collection assembly when the device is worn.

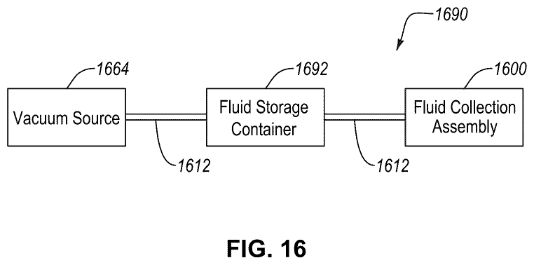

In an embodiment, not shown, the conduit 112 may be at least partially disposed in the chamber 106. The conduit 112 may be used to remove fluid form the chamber 106. The conduit 112 (e.g., a tube) includes the inlet 126 and an outlet (not shown) positioned downstream from the inlet 126. The outlet may be operably coupled to a suction source, such as a vacuum pump for withdrawing the bodily fluids form the chamber through the conduit 112. The conduit 112 fluidly couples the chamber 106 with the fluid storage container (fluid storage container 1692 shown in FIG. 16 or the vacuum source (vacuum source 1664 shown in FIG. 16 The conduit 112 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 112 may include silicon or latex. In some examples, the conduit 112 may include one or more portions that are resilient, such as to by having one or more of a diameter or wall thickness that allows the conduit to be flexible.

In an example, the conduit 112 is configured to be at least insertable into the chamber 106. In such an example, the conduit 112 may include one or more markers (not shown) on an exterior thereof that are located to facilitate insertion of the conduit 112 into the chamber 106. For example, the conduit 112 may include one or more markings thereon that are configured to prevent over or under insertion of the conduit 112, such as when the conduit 112 defines an inlet 126 that is configured to be disposed in or adjacent to the reservoir. In another example, the conduit 112 may include one or more markings thereon that are configured to facilitate correct rotation of the conduit 112 relative to the chamber 106. The one or more markings may include a line, a dot, a sticker, or any other suitable marking.

As described in more detail below, the conduit 112 is configured to be coupled to, and at least partially extend between, one or more of the fluid storage container (not shown) and the vacuum source (not shown). In an example, the conduit 112 is configured to be directly connected to the vacuum source (not shown). In such an example, the conduit 112 may extend from the fluid impermeable barrier 102 by at least one foot, at least two feet, at least three feet, or at least six feet. In another example, the conduit 112 is configured to be indirectly connected to at least one of the fluid storage container (not shown) and the vacuum source (not shown). In some examples, the conduit 112 is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 126 and the outlet of the conduit 112 are configured to fluidly couple (e.g., directly or indirectly) the vacuum source (not shown) to the chamber 106 (e.g., the reservoir). As the vacuum source (FIG. 16) applies a vacuum/suction in the conduit 112, the bodily fluids in the chamber 106 may be drawn into the inlet 126 and out of the fluid collection assembly 100 via the conduit 112. In some examples, the conduit 112 may be frosted or opaque (e.g., black) to obscure visibility of the bodily fluids therein.

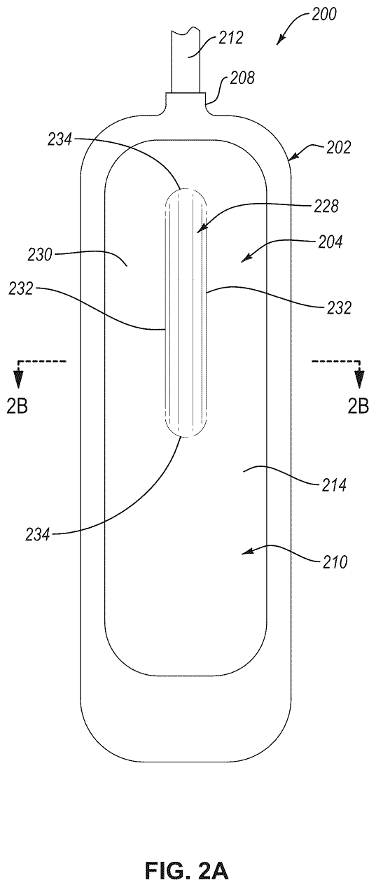

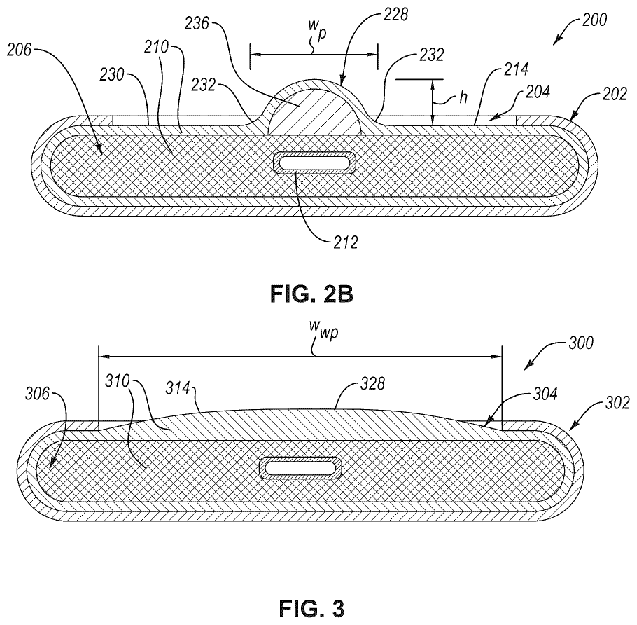

The fluid collection assemblies disclosed herein may include a contact surface that is not substantially flat. For example, FIG. 2A is a top plan view of a fluid collection assembly 200 including a contact surface 214 that is not flat, according to an embodiment. FIG. 2B is a cross-sectional view of the fluid collection assembly 200 taken along line 2B-2B, as shown in FIG. 2A, according to an embodiment. Except as otherwise disclosed herein, the fluid collection assembly 200 may be the same or substantially similar to any of the fluid collection assemblies disclosed herein. For example, the fluid collection assembly 200 may include a fluid impermeable barrier 202 defining an opening 204, a chamber 206, and a fluid outlet 208. The fluid collection assembly 200 may also include a porous material 210 defining the contact surface 214 that extends across the opening 204 and a conduit 212.

As previously discussed, the contact surface 214 is not substantially flat. Instead, the contact surface 214 exhibits a protrusion 228. The sharp protrusion 228 extends from the rest of the contact surface 214 and is configured to press against and at least partially fit between the labia folds. As such, the protrusion 228 may prevent the formation of or minimize the likelihood that the labia folds form passageways through which the bodily fluids may leak. Further, the protrusion 228 may maintain the position of the fluid collection assembly 200 relative to the vaginal region even when the individual moves. Thus, the protrusion 228 may be movement enhancing feature of the fluid collection assembly 200.

The protrusion 228 exhibits a height h and a width Wp. The height h and the width Wp of the protrusion 228 are selected to allow the protrusion 228 to fit between the labia folds of the individual. In an example, the height h may be about 25 mm or less or less, about 20 mm or less or less, about 15 mm or less, about 10 mm or less, about 5 mm or less, or in ranges of about 5 mm to about 10 mm, about 7.5 mm to about 12.5 mm, about 10 mm to about 15 mm, about 12.5 mm to about 17.5 mm, about 15 mm to about 20 mm, about 17.5 mm to about 22.5 mm, or about 20 mm to about 25 mm. In an example, the width Wp is about 50 mm or less, about 40 mm or less, about 30 mm or less, about 20 mm or less, about 10 mm or less, or in ranges of about 10 mm to about 20 mm, about 15 mm to about 25 mm, about 20 mm to about 30 mm, about 25 mm to about 35 mm, about 30 mm to about 40 mm, about 35 mm to about 45 mm, or about 40 mm to about 50 mm. The protrusion 228 exhibiting any of the above height h and width Wp may be sufficient to press against the labia folds and, in some embodiments, may be at least partially positionable between the labia folds. It is noted that the width Wp may be greater than 50 mm, for example when the protrusion 228 tapers (as shown), without affecting the protrusions 228 ability to be at least partially positionable between the labia folds.

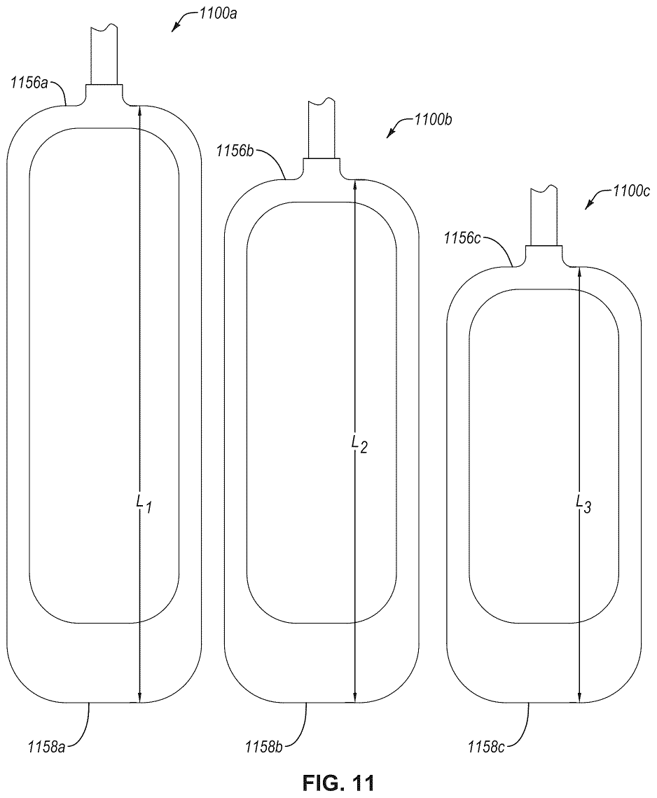

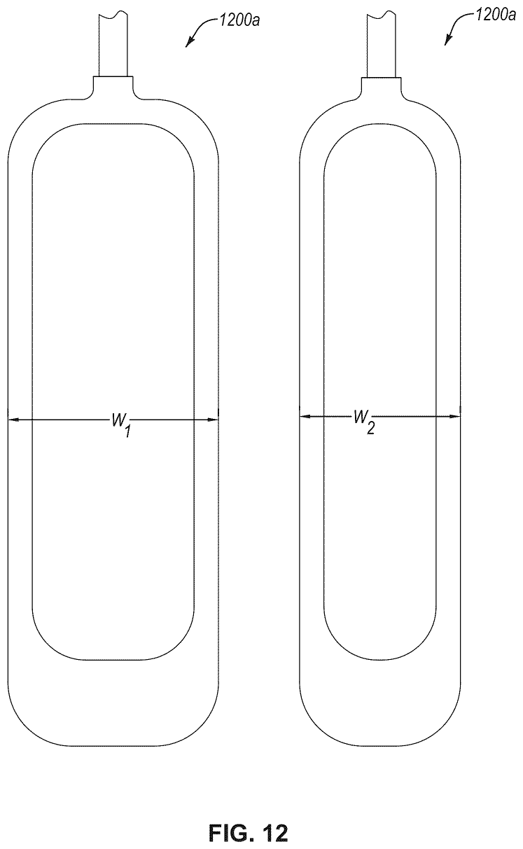

The height h and the width Wp of the protrusion 228 may be selected based on a number of factors. In an embodiment, the height h of the protrusion 228 may be selected based on the width Wp of the protrusion 228. For example, the height h and the Width Wp of the protrusion 228 may be selected such that the protrusion 228 exhibits a generally semi-circular shape (e.g., the height h is about half the width Wp) or such that the height h is greater than half the width Wp since a protrusion 228 exhibiting such heights h and widths Wp may be better able to fit between the labia folds than a protrusion 228 exhibiting a height h that is less than half the width Wp. In another example, the height h and the Width Wp of the protrusion 228 may be selected such that the height h is less than half the width Wp since such a protrusion 228 may be more comfortable than other protrusions. In an embodiment, the height h and the width Wp may be selected based on the size of the individual and/or the size of the fluid collection assembly 200 (as will be discussed in more detail with regards to FIGS. 11 and 12)

In an embodiment, the contact surface 214 includes the protrusion 228 and a flat portion 230. The contact surface 214 may exhibit the benefits of the protrusion 228 and the flat surfaces discussed above when the contact surface 114 includes both the protrusion 228 and the flat portion 230. For example, the protrusion 228 may prevent the formation of passageways through which the bodily fluids may leak and may inhibit the fluid collection assembly 200 from shifting while the individual moves. The flat portion 230 may mitigate the effect of the fluid collection assembly 200 laterally shifting. The flat portion 230 at least partially encloses the protrusion 228. In an example, the flat portion 230 may enclose at least one of the lateral sides 232 of the protrusion 228. In such an example, the flat portion 230 may mitigate the effects of laterally shifting the fluid collection assembly 200. In an example, the flat portion 230 may enclose at least one of the longitudinal sides 234 of the protrusion 228 (e.g., the protrusion 228 does not extend the whole length of the opening 204). In such an example, the protrusion 228 may be configured to be only positioned against or between the labia folds since the protrusion 228 may be uncomfortable when the protrusion 228 presses against other portions of the vaginal region.

In an embodiment, referring to FIG. 2B, the protrusion 228 may be formed may including an additional material 236 in the porous material 210. The additional material 236 increases the thickness of the porous material 210 thereby forming the protrusion 228. In an example, as illustrated, the additional material 236 is positioned between the fluid permeable membrane 222 and the fluid permeable support 224, thereby allowing the fluid permeable membrane 222 to contact the vaginal region. However, it is noted that the additional material 236 may be positioned between the fluid permeable support 224 and the conduit 212 or may be positioned on the fluid permeable membrane 222 such that the additional material 236 contacts the vaginal region. The additional material 236 may be formed from any of the porous materials disclosed herein thereby allowing the additional material 236 to receive bodily fluids and direct the bodily fluids towards the fluid reservoir (not shown). In an embodiment, the protrusion 228 may be formed by increasing the thickness of the fluid permeable membrane 222 and/or the fluid permeable support 224.

In an embodiment, any of the fluid collection assemblies disclosed herein may include a protrusion that extends substantially completely across the opening thereof. FIG. 3 is a cross-sectional view of a fluid collection assembly 300 that includes a protrusion 328 that extends substantially completely across the opening 304 thereof, according to an embodiment. Except as otherwise disclosed herein, the fluid collection assembly 300 may be the same or substantially similar to any of the fluid collection assemblies disclosed herein. For example, the fluid collection assembly 300 may include a fluid permeable barrier 302 defining the opening 304 and a chamber 306. The fluid collection assembly 300 also includes a porous material 310 disposed in the chamber 306 that forms a contact surface 314.

The contact surface 314 includes the protrusion 328. Unlike the protrusion 228 illustrated in FIGS. 2A and 2B, the protrusion 328 may exhibit a relatively wide width Wwp that is greater than 50 mm. For example, as shown, the protrusion 328 may exhibit a wide width Wwp that extends substantially completely across the opening 304. The wide width Wwp may prevent the protrusion 328 from being positionable between the labia folds. However, the protrusion 328 may still press against the labia folds to minimize the passageways formed by the labia folds. Thus, the protrusion 328 may be a movement enhancing feature. Also, the protrusion 328 may be more comfortable than the protrusion 228 due to the wider width of the protrusion 328 relative to the protrusion 228.

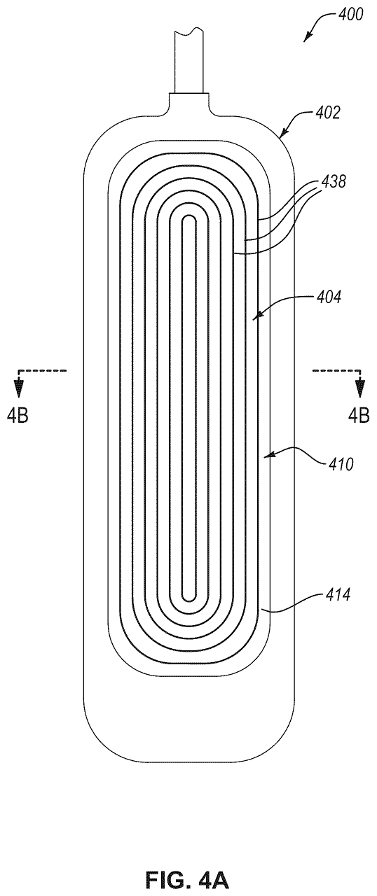

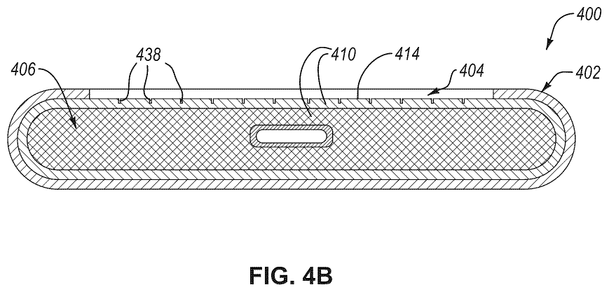

FIG. 4A is a top plan view of a fluid collection assembly 400 that includes one or more grooves 438 formed in the porous material 410 that are movement enhancing features, according to an embodiment. FIG. 4B is a cross-sectional view of the fluid collection assembly 400 taken along line 4B-4B shown in FIG. 4B, according to an embodiment. Except as otherwise disclosed herein, the fluid collection assembly 400 may be the same or substantially similar to any of the fluid collection assemblies disclosed herein. For example, the fluid collection assembly 400 may include a fluid impermeable barrier 402 defining an opening 404 and a chamber 406. The fluid collection assembly 400 may also include at least one porous material 410 disposed in the chamber 406 that defines a contact surface 414.

As previously discussed, the porous material 410 defines one or more grooves 438. The grooves 438 are movement enhancing features that are better able to grip the vaginal region since the grooves 438 increase the friction between the contact surface 414 and the vaginal region. Thus, the grooves 438 inhibit movement of the fluid collection assembly 400 when the individual moves. Additionally, the grooves 438 increase the surface area of the contact surface 414 thereby increasing the amount of bodily fluids that contact and may flow through the contact surface 414 at any given time.

In an embodiment, the grooves 438 are formed by decreasing the thickness of at least a portion of the porous material 410. For example, when the porous material 410 includes a fluid permeable membrane 422, the grooves 438 may be formed by selectively decreasing the thickness of portions of the fluid permeable membrane 422. In an embodiment, the grooves 438 may be formed by compressing a selected portion of the porous material 410. In such an embodiment, the grooves 438 may be formed by selectively sewing seams into porous material 410, selectively compressing portions of the porous material 410 and using an adhesive or heat (e.g., melting a portion of the porous material 410) to maintain the compressed shape, or any other suitable technique for compressing a selected portion of the porous material 410. In an embodiment, the grooves 438 are formed between ridges that extend from the rest of the porous material 410, wherein the grooves 438 are the space between adjacent ridges.

In an embodiment, as shown, the grooves 438 may form concentric annular shapes. The concentric annular shapes may be generally oval (as shown), generally circular, an irregular shape (e.g., an irregular curved shape), or any other suitable shape. In an embodiment, the grooves 438 may include one or more connected or non-connected lines, such as one or more curved lines. In an embodiment, the grooves 438 may form a network of interconnected grooves 438.

The grooves 438 may exhibit a depth and a width. The depth and the width of the grooves 438 may be about 0.1 mm to about 0.3 mm, about 0.2 mm to about 0.4 mm, about 0.3 mm to about 0.5 mm, about 0.4 mm to about 0.6 mm, about 0.5 mm to about 0.75 mm, about 0.7 mm to about 1 mm, about 0.75 mm to about 1.25 mm, about 1 mm to about 1.5 mm, about 1.25 mm to about 1.75 mm, about 1.5 mm to about 2 mm, about 1.75 mm to about 2.5 mm, or about 2 mm to about 3 mm. The depth and the width of the grooves 438 may be selected based on the size of the contact surface 414 and the number of grooves 438 formed in the contact surface 414.

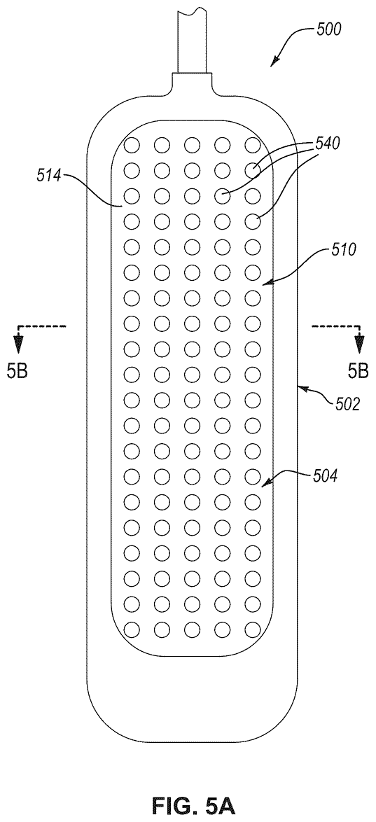

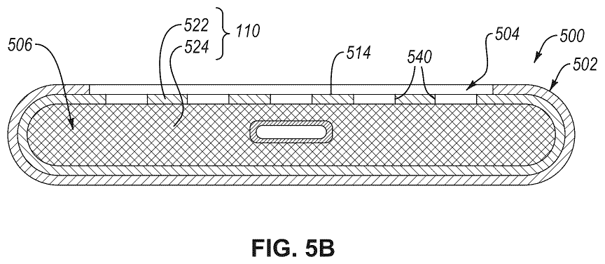

The movement enhancing features of the fluid collection assemblies disclosed herein may include perforations formed in the porous material instead of or in conjunction with any of the other movement enhancing features disclosed herein. FIG. 5A is a top plan view of a fluid collection assembly 500 that one or more perforations 540 formed in the porous material 510 that are movement enhancing features, according to an embodiment. FIG. 5B is a cross-sectional view of the fluid collection assembly 500 taken along line 5B-5B shown in FIG. 5B, according to an embodiment. Except as otherwise disclosed herein, the fluid collection assembly 500 may be the same or substantially similar to any of the fluid collection assemblies disclosed herein. For example, the fluid collection assembly 500 may include a fluid impermeable barrier 502 defining an opening 504 and a chamber 506. The fluid collection assembly 500 may also include at least one porous material 510 disposed in the chamber 506 that defines a contact surface 514.

As previously discussed, the porous material 510 defines one or more perforations 540. The perforations 540 are movement enhancing features because the perforations 540 allow the contact surface 514 to better grip the vaginal region since the perforations 540 increase the friction between the contact surface 514 and the vaginal region. Thus, the perforations 540 inhibit movement of the fluid collection assembly 500 when the individual moves. Additionally, the perforations 540 increase the surface area of the contact surface 514, thereby increasing the amount of bodily fluids that contact and may flow through the contact surface 514 at any given time.

The perforations 540 are distinct from the inherent porosity of the porous material 510. For example, the perforations 540 may exhibit an average maximum dimension (e.g., diameter) that is about 0.25 mm or greater, such as about 0.5 mm or greater, about 0.75 mm or greater, about 1 mm or greater, about 1.5 mm or greater, about 2 mm or greater, or in ranges of about 0.25 mm to about 0.75 mm, about 0.5 mm to about 1 mm, about 0.75 mm to about 1.5 mm, or about 1 mm to about 2 mm. As such, the perforations 540 are easily visible. Meanwhile, the average maximum dimension of the inherent porosity of the porous material 510 are significantly less than the perforations 540, such as exhibiting an average maximum dimension that is smaller than the average maximum dimensions of the perforations 540 by about 50% or more, about 75% or more, about 100% or more, about 150% or more, about 200% or more, about 300% or more, about 400% or more, about 500% or more, about 750% or more, or about 1000% or more.

As previously discussed, the porous material 510 may include a fluid permeable membrane 522 and a fluid permeable support 524. In an embodiment, as illustrated, at least one of the perforations 540 may extend completely through the fluid permeable membrane 522. In such an embodiment, the perforations 540 may allow the bodily fluids to more quickly flow through the fluid permeable membrane 522 thereby increasing the rate at which the porous material 510 may receive the bodily fluids. In an embodiment, at least one of the perforations 540 only extends partially through the fluid permeable membrane 522, such as recesses or dimples formed in the fluid permeable membrane 522. In such an embodiment, the perforations 540 may still allow the bodily fluids to more quickly flow through the fluid permeable membrane 522. In an embodiment, at least one of the perforations 540 may extend completely through the fluid permeable membrane 522 and at least partially through the fluid permeable support 524. In such an embodiment, the perforations 540 may allow the bodily fluids to flow more quickly through the porous material 510 but may also increase the likelihood that the bodily fluids leak from the chamber 506.

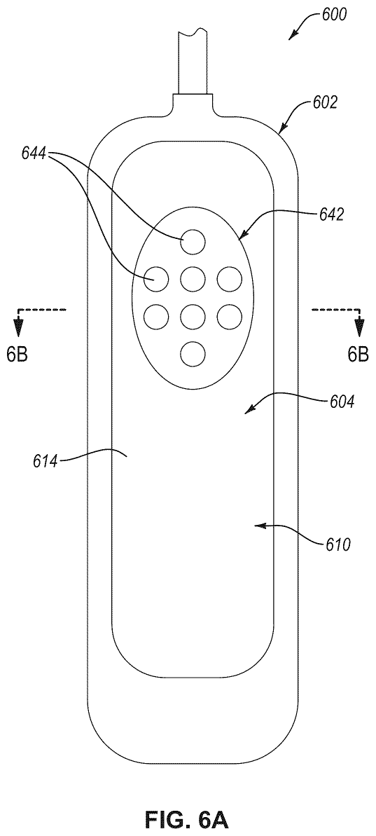

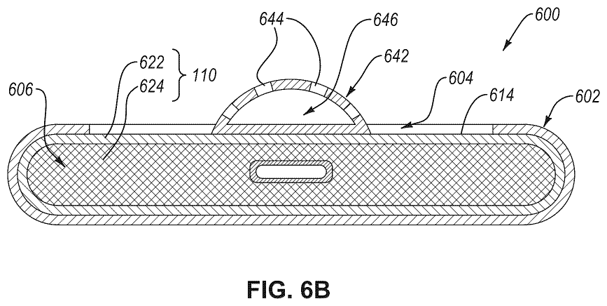

The movement enhancing features illustrated and discussed in FIGS. 2A-5B are formed in the porous material thereof. However, the movement enhancing features may be formed from a fluid permeable element that is distinct from and attached to the porous material. FIG. 6A is a top plan view of a fluid collection assembly 600 that includes a fluid permeable element 642, according to an embodiment. FIG. 6B is a schematic cross-sectional view of the fluid collection assembly 600 taken along line 6B-6B shown in FIG. 6A, according to an embodiment. Except as otherwise disclosed herein the fluid collection assembly 600 is the same or substantially similar to any of the fluid collection assemblies disclosed herein. For example, the fluid collection assembly 600 may include a fluid impermeable barrier 602 defining an opening 604 and a chamber 606. The fluid collection assembly 600 may also include a porous material 610 disposed in the chamber 606 that defines a contact surface 614 extending across the opening 604.

As previously discussed, the fluid collection assembly 600 includes a fluid permeable element 642 that is distinct from and attached to the porous material 610. In an embodiment, the fluid permeable element 642 may be harder and/or more rigid than the porous material 610. As such, the fluid permeable element 642 may be less likely to compress or otherwise deform compared to the porous material 610 since compressing or otherwise deforming the porous material 610 may limit the functionality of the porous material 610. For example, referring to the fluid collection assembly 200 illustrated in FIGS. 2A and 2B, the protrusion 228 may be compressed while attempting to position the protrusion 228 between the labia folds of the individual which may diminish the movement enhancing features of the protrusion 228 and limit the protrusion's 228 ability to receive bodily fluids. However, referring back to FIGS. 6A and 6B, the fluid permeable element 642 may be more easily positioned between the labia folds without compressing compared to the fluid permeable element 642.

In an embodiment, the fluid permeable element 642 may be harder and/or more rigid than the porous material 610 because the fluid permeable element 642 is formed from a material exhibiting a Young's modulus that is greater than the Young's modulus of the material that forms at least a portion of the porous material 610. Examples of such materials include silicone. In an embodiment, the fluid permeable element 642 may be harder and/or more rigid than the porous material 610 because the fluid permeable element 642 is formed from a non-porous material or exhibits a porosity that is less than the porous material 610.

The fluid permeable element 642 may define one or more apertures 644 extending therethrough, such as when the fluid permeable element 642 is formed from a non-porous material. The apertures 644 may allow the bodily fluids to flow through the fluid permeable element 642.

In an embodiment, the fluid permeable element 642 defines a hollowed region 646. The hollowed region 646 may be substantially unoccupied space (as shown) or may be occupied by an additional porous material. Similar to the porous material 610, the additional porous material may direct the flow of any bodily fluids that enter the hollowed region 646 towards the porous material 610 thereby inhibiting leaks. The additional porous material may be formed from any of the porous materials disclosed herein. For example, the additional porous material may be formed from the same material as at least a portion of the porous material 610.

The fluid permeable element 642 may be attached to the porous material 610 using any suitable method. In an embodiment, as shown, the fluid permeable element 642 is attached directly to the contact surface 614 such that the fluid permeable element 642 directly contacts the vaginal region during operation. In such an embodiment, the fluid permeable element 642 may be attached to the porous material 610 via an adhesive, stitching, heat staking, or any other suitable technique. In an embodiment, the fluid permeable element 642 may be attached to the porous material 610 by disposing at least a portion of the fluid permeable element inside the porous material 610. For example, the fluid permeable element 642 may be disposed between the fluid permeable membrane 622 and the fluid permeable support 624 or the porous material 610 may define a pocket (not shown) that is configured to receive the fluid permeable element 642. As such, the porous material 610 contacts the vaginal region which may make the fluid collection assembly 600 more comfortable to use than if the fluid permeable element 642 directly contacts the vaginal region. It is noted that the fluid permeable element 642 may still be attached to the porous material 610 (e.g., via an adhesive, stitching, heat staking, etc.) even when the fluid permeable element 642 is disposed in the porous material 610 to prevent the fluid permeable element 642 from moving within the porous material 610.

The fluid permeable element 642 may form and/or include any of the movement enhancing features disclosed herein. For example, the fluid permeable element 642 may form a flat surface, a protrusion exhibiting a width of about 25 mm or less, a protrusion exhibiting a width of about 25 mm or more, one or more grooves, or one or more perforations therein.

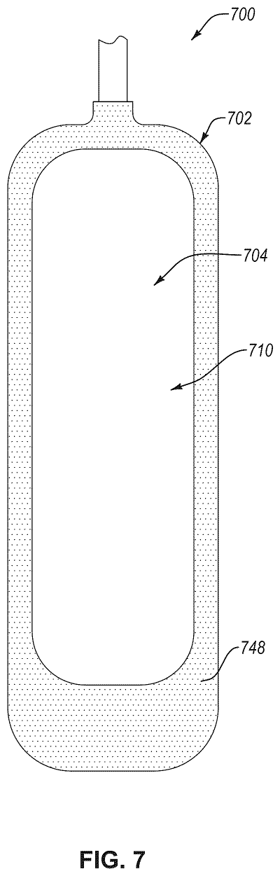

The fluid impermeable barriers of any of the fluid collection assemblies disclosed herein may include a movement enhancing feature. FIG. 7 is a top plan view of a fluid collection assembly 700 that includes a fluid impermeable barrier 702 having one or more movement enhancing features, according to an embodiment. Except as otherwise disclosed herein, the fluid collection assembly 700 is the same or substantially similar to any of the fluid collection assemblies disclosed herein. For example, the fluid impermeable barrier 702 of the fluid collection assembly 700 may define an opening 704 and a chamber (not shown). The fluid collection assembly 700 may also include a porous material 710 disposed in the chamber.

The fluid impermeable barrier 702 includes a textured surface 748. The textured surface 748 of the fluid impermeable barrier 702 allows the fluid impermeable barrier 702 to better grip the vaginal region thereby inhibiting movement of the fluid collection assembly 700 relative to the vaginal region when the individual moves. In an embodiment, as shown, the textured surface 748 of the fluid impermeable barrier 702 includes a plurality of bumps extending from the rest of the fluid impermeable barrier 702. The average diameter of the plurality of bumps may be selected to be about 3 mm or less, about 2 mm or less, about 1 mm or less, about 0.5 mm or less, about 0.1 mm or less, or in ranges of about 0.1 mm to about 1 mm, about 0.5 mm to about 2 mm, or about 1 mm to about 3 mm. In an embodiment, the textured surface 748 may include a plurality of grooves formed therein that extend partially through the fluid impermeable barrier 702. The grooves formed in the fluid impermeable barrier 702 may be the same or substantially similar to any of the grooves disclosed herein. In an embodiment, the textured surface 748 may include a plurality of perforations that extend partially through the fluid impermeable barrier 702. The perforations formed in the fluid impermeable barrier 702 may be the same or substantially similar to any of the perforations disclosed herein.

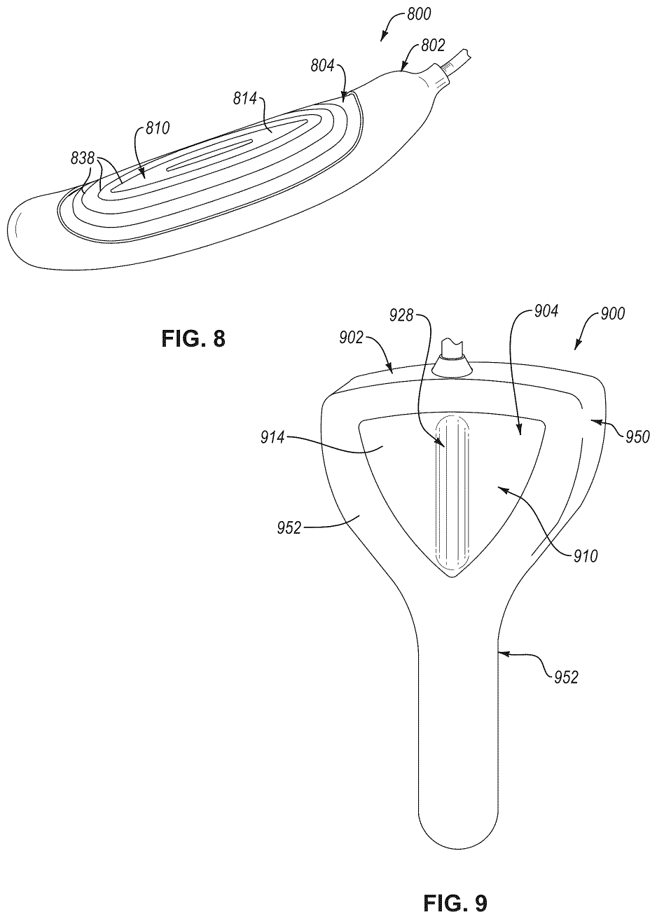

The fluid collection assemblies illustrated in FIGS. 1A-7 exhibit an elongated generally flat shape having a relatively small thickness. As discussed above, fluid collection assemblies exhibiting such shapes exhibit several benefits. However, it is noted that the fluid collection assemblies disclosed herein may exhibit different shapes than the shape illustrated in FIGS. 1A-7. For example, FIGS. 8 and 9 are isometric view of fluid collection assemblies exhibiting different shapes, according to different embodiments. Except as otherwise disclosed herein, the fluid collection assemblies illustrated in FIGS. 8 and 9 are the same or substantially similar to any of the fluid collection assemblies disclosed herein.

Referring to FIG. 8, the fluid collection assembly 800 includes a fluid impermeable barrier 802 defining an opening 804 and a chamber (not shown). The fluid collection assembly 800 also includes a porous material 810 disposed in the chamber that defines a contact surface 814. The fluid collection assembly 800 exhibits a generally cylindrical shape (e.g., a curved generally cylindrical shape). The generally cylindrical shape of the fluid collection assembly 800 allows the fluid collection assembly 800 to fit between the thighs of the individual. Further, due to the cylindrical shape of the fluid collection assembly 800, the contact surface 814 may exhibit a curvature that allows the contact surface 814 to press against the labia folds and may be positionable between the labia folds.

The fluid collection assembly 800 may include any of the movement enhancing features disclosed herein. In an example, the porous material 810 may form a protrusion exhibiting a radius that is less than the radius of the rest of the porous material 810 which, similar to the protrusion 228 of FIGS. 2A and 2B, may allow the protrusion to be more positionable between the labia folds that the rest of the porous material 810. In an example, the porous material 810 may extend slightly out from the opening 804 similar to the protrusion 328 of FIG. 3. In an example, as illustrated, the porous material 810 may define one or more grooves 838 extending at least partially therethrough, similar to the grooves 438 of FIGS. 4A and 4B. In an example, the porous material 810 may define one or more perforations extending at least partially therethrough that are similar to the perforations 540 of FIGS. 5A and 5B. In an example, the fluid collection assembly 800 may include a fluid permeable element that is attached to the porous material 810 that is similar to the fluid permeable element 642 of FIGS. 6A and 6B. In an example, the fluid impermeable barrier 802 may include a textured surface similar to the textured surface 748 of FIG. 7.

Referring to FIG. 9, the fluid collection assembly 900 includes a fluid impermeable barrier 902 defining an opening 904 and a chamber (not shown). The fluid collection assembly 900 also includes a porous material 910 disposed in the chamber that defines a contact surface 914. The fluid collection assembly 900 includes a fluid receiving portion 950 that includes the portions of the fluid impermeable barrier 902 that defines the opening 904. The urinary collection assembly 900 also includes a longitudinal portion 952 (e.g., elongated portion) extending from the fluid receiving portion 950 formed by a portion of the fluid impermeable barrier 902. The longitudinal portion 952 is configured to be positioned adjacent to or near the perineum of the individual (e.g., positioned between the thighs of the individual).

The fluid receiving portion 950 exhibits a shape that allows the fluid receiving portion 950 to be positioned adjacent to the urethral opening. Generally, a front surface 954 of the fluid receiving portion 950 and the contact surface 914 exhibits a shape that allows the fluid receiving portion 950 to abut the urethral opening. For example, the front surface 954 and the contact surface 914 may exhibit a generally planar topography or may have a concave curved topography which allows the front surface 954 and the contact surface 914 to conform to the vaginal region since the vaginal region is generally planar or has a convex curvature.

The fluid receiving portion 950 may also exhibit a shape that is configured to increase the size of the opening 904 (e.g., the opening 904 generally corresponds to the shape of the fluid receiving portion 950). For example, increasing the size of the fluid receiving portion 950 may allow the fluid receiving portion 950 to define a larger opening 904. The larger opening 904 allows the opening 904 to receive bodily fluids that is emitted from a urethral opening, even when the individual moves in a manner that causes the fluid collection assembly 900 to shift (i.e., the larger opening 904 is a movement enhancing feature). Further, the larger opening 904 allows the opening 904 to receive bodily fluids that would have otherwise leaked between the fluid impermeable barrier 902 and the individual.

In an embodiment, the fluid receiving portion 950 may exhibit a generally triangular shape. The generally triangular shape may allow the fluid receiving portion 950 to correspond to the shape of the groin that surrounds the vaginal region since the groin exhibits a generally triangular shape, especially when the thighs of the individual contact each other. As such, the generally triangular shape of the fluid receiving portion 950 may allow the fluid receiving portion 950 to exhibit the maximum possible size without uncomfortably pressing into the individual. Further, the generally triangular shape of the fluid receiving portion 950 may be movement enhancing features because the generally triangular shape of the fluid receiving portion 950 may substantially occupy the groin thereby preventing the fluid collection assembly 900 from shifting even when the individual moves.

It is noted that the fluid receiving portion 950 may exhibit different shapes than the generally triangular shape illustrated in FIG. 9. For example, the fluid receiving portion 950 may exhibit a generally circular shape, a generally rectangular shape, or any other suitable shape.

The longitudinal portion 952 is distinguishable from the fluid receiving portion 950 based on the widths thereof. For example, in the illustrated embodiment, the width of the fluid receiving portion 950 generally decreases from the maximum width thereof to the longitudinal portion 952 while the width of the longitudinal portion 952 remains substantially constant from the intersection of the fluid receiving portion 950 and the longitudinal portion 952 along at least a portion of a length of the longitudinal portion 952. This may allow the longitudinal portion 952 to fit between the thighs of the individual.

The fluid collection assembly 900 may include any of the movement enhancing features disclosed herein. In an example, as shown, the porous material 910 may form a protrusion 928 similar to the protrusion 228 of FIGS. 2A and 2B. In an example, the porous material 910 may extend slightly out from the opening 904 similar to the protrusion 328 of FIG. 3. In an example, the porous material 910 may define one or more grooves extending at least partially therethrough, similar to the grooves 438 of FIGS. 4A and 4B. In an example, the porous material 910 may define one or more perforations extending at least partially therethrough that are similar to the perforations 540 of FIGS. 5A and 5B. In an example, the fluid collection assembly 900 may include a fluid permeable element that is attached to the porous material 910 that is similar to the fluid permeable element 642 of FIGS. 6A and 6B. In an example, the fluid impermeable barrier 902 may include a textured surface similar to the textured surface 748 of FIG. 7.

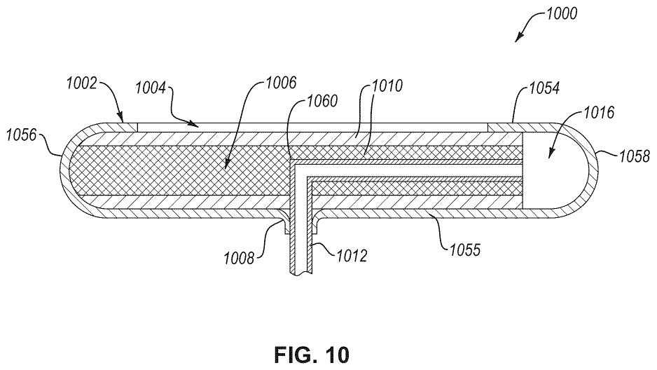

The fluid collection assemblies illustrated in FIGS. 1A-9, the conduit is illustrated as extending away from a distal end of the fluid collection assembly. As such, when the conduit is straight, the conduit and the rest of the fluid collection assembly generally lies in the same plane. However, when the conduit and the rest of the fluid collection assembly are in the same plane, the conduit may act as a lever. As such, any movement in the conduit caused by the individual moving may cause the fluid collection assemblies illustrated in FIGS. 1A-9 to shift since, as a lever, the conduit may act as a force multiplier. As such, in some embodiments, the movement enhancing features of the fluid collection assemblies may include the conduit, when straight, not being in the same plane as the rest of the fluid collection assembly. FIG. 10 is a cross-sectional view of a fluid collection assembly 1000 including a conduit 1012 extending from a back surface 1055 of the fluid impermeable barrier 1002, according to an embodiment. Except as otherwise disclosed herein, the fluid collection assembly 1000 is the same or substantially similar to any of the fluid collection assemblies disclosed herein. For example, the fluid collection assembly 1000 may include a fluid impermeable barrier 1002 that defines an opening 1004, a chamber 1006, a fluid outlet 1008, and a substantially unoccupied fluid reservoir 1016. The fluid collection assembly 1000 may also include a porous material 1010 disposed in the chamber 1006.

The fluid impermeable barrier 1002 includes a distal end 1056 and a proximal end 1058 longitudinally spaced from the distal end 1056. In some embodiments, as shown, the proximal end 1058 may define the fluid reservoir 1016. The fluid impermeable barrier 1002 also includes a front surface 1054 and a back surface 1055 opposite the front surface 1054. The front surface 1054 and the back surface 1055 extend between the distal end 1056 and the proximal end 1058. The front surface 1054 defines the opening 1004 and the back surface 1055 defines the fluid outlet 1008.

The fluid collection assembly 1000 includes a conduit 1012. The conduit 1012 is secured to and extends from the fluid outlet 1008. Since the fluid outlet 1008 is defined by the back surface 1055 of the fluid impermeable barrier 1002, the conduit 1012 extends from the back surface 1055 of the fluid impermeable barrier 1002 instead of the distal end 1056, as shown in FIGS. 1A-9. As such, the conduit 1012 is less likely to act as a lever than the conduits illustrated in FIGS. 1A-9 since the conduit 1012, when straight, does not extend in substantially the same plane as the rest of the fluid collection assembly 1000.

In an embodiment, as shown, the conduit 1012 extends at about a perpendicular angle relative to the portion of the back surface 1055 that defines the fluid outlet 1008. However, in an embodiment, the conduit 1012 may extend from the portion of the back surface 1055 that defines the fluid outlet 1008 at an angle that is about 5° to about 89° (e.g., about 5° to about 45°, about 30° to about 60°, or about 45° to about 89°) or about 91° to about 175° (e.g., about 91° to about 135°, about 120° to about 150°, or about 135° to about 175°).

The conduit 1012 may extend into the chamber 1006. The conduit 1012 may include a bend 1060 therein when the conduit 1012 extends into the chamber 1006 thereby allowing the conduit 1012 to extend further into the chamber 1006. For example, the bend 1060 may allow the conduit 1012 to extend from the fluid outlet 1008 to a location that is adjacent to or within the fluid reservoir 1016. In an embodiment, the bend 1060 is formed in the conduit 1012. In an embodiment, the bend 1060 is a joint that connects a first portion of the conduit 1012 to a second portion of the conduit 1012 that is distinct from the first portion of the conduit 1012. In an embodiment, the bend 1060 is a joint that allows the angle that the conduit 1012 extends from the back surface 1055 to vary.

The vaginal region may exhibit different sizes depending on the individual. A fluid collection assembly that is not configured for the particular size of the vaginal region is more likely shift when the individual moves. As such, in some embodiments, the movement enhancing features of the fluid collection assemblies disclosed herein may include providing a plurality of fluid collection assemblies wherein at least one of the plurality of fluid collection assemblies exhibit a different size. The different size of the plurality of fluid collection assemblies allows a fluid collection assembly to be selected that is better configured for the particular size of the vaginal region than the rest of the fluid collection assemblies. The different sizes of the plurality of fluid collection assemblies may include at least one of a plurality of fluid collection assemblies exhibiting at least one of different lengths, different widths, different thicknesses, or different shapes.

FIG. 11 is a top plan view of a plurality of fluid collection assemblies exhibiting different lengths, according to an embodiment. Except as otherwise disclosed herein, each of the plurality of fluid collection assemblies may be the same or substantially similar to any of the fluid collection assemblies disclosed herein. For example, each of the plurality of fluid collection assemblies are illustrated as being substantially similar to the fluid collection assembly 100 illustrated in FIG. 1. However, it is noted that at least one of the plurality of fluid collection assemblies disclosed herein may be similar to any of the other fluid collection assemblies disclosed herein.

In the illustrated embodiment, the plurality of fluid collection assemblies includes a first fluid collection assembly 1100a, a second fluid collection assembly 1100b, and a third fluid collection assembly 1100c. The first fluid collection assembly 1100a exhibits a first length L₁ measured from a distal end 1156a to a proximal end 1158a of the fluid impermeable barrier 1102a thereof. The second fluid collection assembly 1100b exhibits a second length L₂ measured from a distal end 1156b to a proximal end 1158b of the fluid impermeable barrier 1102b thereof. The third fluid collection assembly 1100c exhibits a third length L₃ measured from a distal end 1156c to a proximal end 1158c of the fluid impermeable barrier 1102c thereof. The first length L₁ is greater than the second length L₂ and the second length L₂ is greater than the third length L₃.

The individual or a medical practitioner may select one of the first fluid collection assembly 1100a, second fluid collection assembly 1100b, or the third fluid collection assembly 1100c based on the size of the vaginal region. For example, generally, the individual or the medical practitioner may select the fluid collection assembly with the longest length since increasing the length of the fluid collection assembly may decrease the amount of bodily fluids that leaks. However, the fluid collection assembly is more likely to shift when the individual moves if the length is too great. For example, movement of the individual may cause the glutes and/or abdominal muscles to flex and move which may cause the fluid collection assembly to shift if the fluid collection assembly is adjacent to the glutes and/or abdominal muscles. Thus, to allow increased movement of the individual, the individual or the medical practitioner may select one of the first fluid collection assembly 1100a, second fluid collection assembly 1100b, or the third fluid collection assembly 1100c such that the fluid collection assembly is not adjacent to at least one of the glutes and/or the abdominal muscles.

FIG. 12 is a top plan view of a plurality of fluid collection assemblies exhibiting different widths, according to an embodiment. Except as otherwise disclosed herein, each of the plurality of fluid collection assemblies may be the same or substantially similar to any of the fluid collection assemblies disclosed herein. For example, each of the plurality of fluid collection assemblies are illustrated as being substantially similar to the fluid collection assembly 100 illustrated in FIG. 1. However, it is noted that at least one of the plurality of fluid collection assemblies disclosed herein may include any of the fluid collection assemblies disclosed herein.

In the illustrated embodiment, the plurality of fluid collection assemblies includes a first fluid collection assembly 1200a and a second fluid collection assembly 1200b. The first fluid collection assembly 1200a exhibits a first width W₁ measured perpendicularly to a longitudinal length thereof. The second fluid collection assembly 1200b exhibits a second width W₂ measured perpendicularly to a longitudinal length thereof. The first width W₁ is greater than the second width W₂.

The individual or a medical practitioner may select one of the first fluid collection assembly 1200a or the second fluid collection assembly 1200b based on the size of the vaginal region. For example, generally, the individual or the medical practitioner may select the fluid collection assembly with the largest width since increasing the width of the fluid collection assembly may decrease the amount of bodily fluids that leaks. However, movement of the thighs may cause the fluid collection assembly to shift or buckle if the width is too large. Thus, to allow increased movement of the individual, the individual or the medical practitioner may select one of the first fluid collection assembly 1200a or the second fluid collection assembly 1200b based on the size of the vaginal region and, in particular, the space between the thighs.

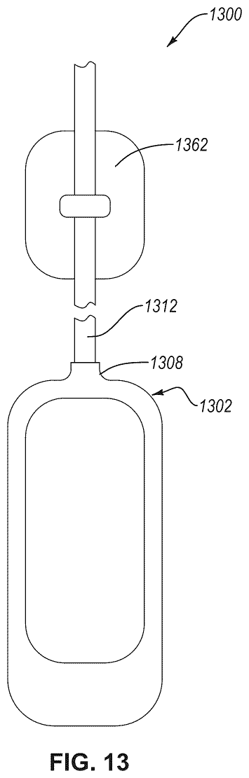

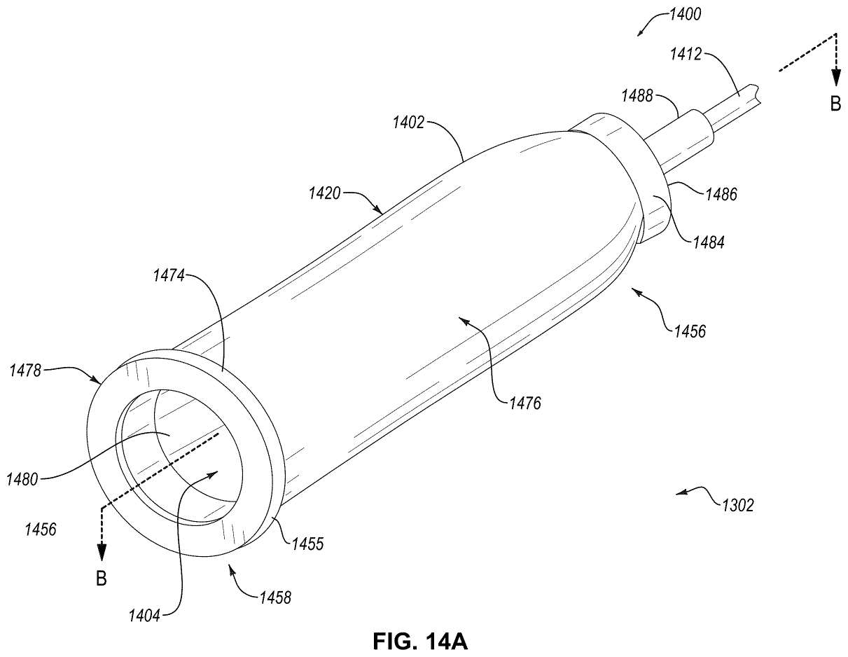

As previously discussed, the conduit of the fluid collection assemblies disclosed herein may act as levers that cause the fluid collection assembly that includes the conduit to shift when the individual using the fluid collection assembly moves. The conduit is most likely to act as a lever when the conduit, when straight, extends in the same plane as the rest of the fluid collection assembly. However, it is noted that the conduit may still act as a lever when the conduit, when straight, does not extend in the same plane as the rest of the fluid collection assembly though not to the extent as when the conduit extends in the same plane as the rest of the fluid collection assembly. However, securing the conduit to a location that is proximate to the fluid outlet (e.g., the thigh or abdominal region of the individual) may help at least partially mitigate the effect of the conduit acting as a lever. FIG. 13 is a top plan view of a fluid collection assembly 1300 that is configured to have the conduit 1312 thereof secured to a location that is proximate to the fluid outlet 1308 thereof, according to an embodiment. Except as otherwise disclosed herein, the fluid collection assembly 1300 is the same or substantially similar to any of the fluid collection assemblies disclosed herein. For example, the fluid collection assembly 1300 may include a fluid impermeable barrier 1302 defining the fluid outlet 1308 and a conduit 1312 secured to the fluid outlet 1308.

A portion of the conduit 1312 that is spaced (i.e., located downstream) from the fluid permeable barrier 1302 may be configured to be secured to a location that is proximate to (e.g., within about 2 feet, within about 1.5 feet, within about 1 foot, within about 9 inches, within about 6 inches, within about 3 inches, or in ranges of about 1 inch to about 6 inches, about 3 inches to about 9 inches, about 6 inches to about 1 foot, about 9 inches to about 1.5 feet, or about 1 foot to about 2 feet) the fluid outlet 1308. In an embodiment, as illustrated, the portion of the conduit 1312 is secured to the location using with a catheter securement device 1362, such as a STATLOCK® catheter securement device available from C. R. Bard, Inc., including but not limited to those disclosed in U.S. Pat. Nos. 6,117,163; 6,123,398; and 8,211,063, the disclosures of which were previously incorporated herein. In an embodiment, the portion of the conduit 1312 is secured to the location using tape or other suitable device.

The fluid collection assemblies shown in FIGS. 1A-13 are examples of female fluid collection assemblies that are configured to collect bodily fluids from females. However, the fluid collection assemblies, systems, and method disclosed herein may include male fluid collection assemblies shaped, sized, and otherwise configured to collection bodily fluids from males (e.g., collect urine from a male urethral opening).

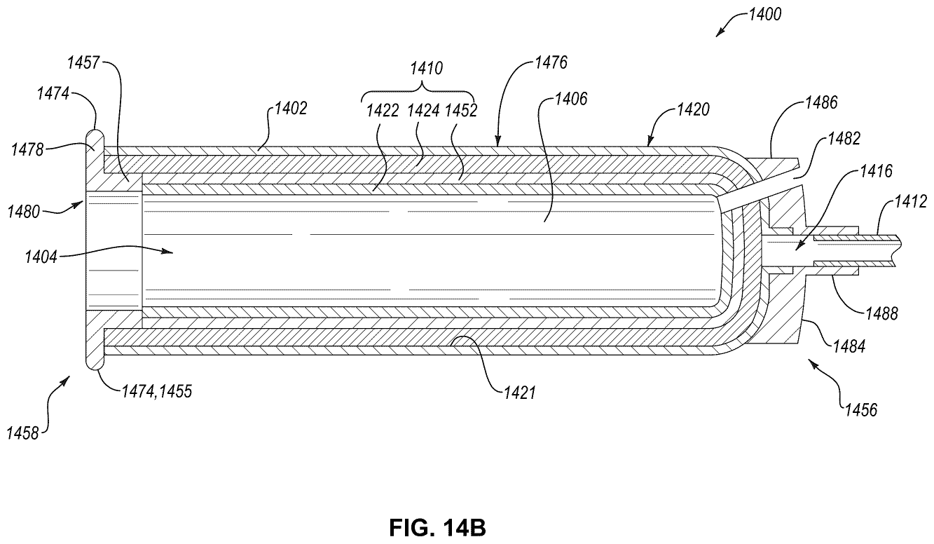

FIG. 14A is an isometric view of a fluid collection assembly 1400 according to an embodiment. FIG. 14B is a cross-sectional view of the fluid collection assembly 1400 of FIG. 14A taken along the plane 14B-14B of FIG. 14A, according to an embodiment. Referring to FIG. 14A and FIG. 14B, the fluid collection assembly 1400 includes a receptacle 1474 and a sheath 1476. The receptacle 1474 is sized, shaped, and made of a material to be coupled to skin that surrounds the male urethral opening and have the male urethral opening positioned therethrough. For example, the receptacle 1474 may include an annular base 1478 that defines an opening 1480 in the receptacle 1474. The annular base 1478 is sized and shaped to be positioned around the male urethral opening (e.g., positioned around and/or over the penis) and the opening 1480 may be configured to have the male urethral opening positioned therethrough. The annular base 1478 may also be sized, shaped, made of a material, or otherwise configured to be coupled (e.g., adhesively attached, such as with a hydrogel adhesive) to the skin around the male urethral opening (e.g., around the penis). In an example, the annular base 1478 may exhibit the general shape or contours of the skin surface that the annular base 1478 is selected to be coupled with. The annular base 1478 may be flexible thereby allowing the annular base 1478 to conform to any shape of the skin surface. The annular base 1478 may include a laterally extending flange 1455. The receptacle 1474 also defines a hollowed region that is configured to receive (e.g., seal against) the sheath 1476. For example, the receptacle 1474 may include a longitudinally extending flange 1457 that extends upwardly from the annular base 1478. The longitudinally extending flange 1457 may be tall enough to prevent the sheath 1476 from being accidentally removed from the receptacle 1474 (e.g., at least 0.25 cm tall, 1 cm tall, at least 2.5 cm tall, or at least 5 cm tall). The receptacle 1474 is located at a proximal end 1458 (with respect to a wearer) of the fluid collection assembly 1400.

The sheath 1476 includes (e.g., may be formed from) a fluid impermeable barrier 1402 that is sized and shaped to fit into the hollowed region of the receptacle 1474. For example, the sheath 1476 may be generally tubular or cup-shaped, as shown. The generally tubular or cup-shaped fluid impermeable barrier 1402 may at least partially define the outer surface 1420 of the sheath 1476. The fluid impermeable barrier 1402 may be similar or identical to any of the fluid impermeable barriers disclosed herein, in one or more aspects. For example, the fluid impermeable barrier 1402 may be constructed of any of the materials disclosed herein for the fluid impermeable barrier 1402. The fluid impermeable barrier 1402 at least partially defines the chamber 1406. For example, the inner surface 1421 of the fluid impermeable barrier 1402 at least partially defines the perimeter of the chamber 1406. The chamber 1406 may be similar or identical to any of the chambers disclosed herein in one or more aspects. For example, the chamber 1406 may at least temporarily retain bodily fluids therein. As shown, the fluid collection assembly 1400 may include at least one porous material 1410 therein. The porous material 1410 may be similar or identical to any of the porous materials disclosed herein in one or more aspects. For example, the porous material 1410 may include one or more of a fluid permeable membrane 1422, a fluid permeable support 1424, or an absorbent layer 1452. The fluid impermeable barrier 1402 may also define an opening 1404 extending through the fluid impermeable barrier 1402 that is configured to have a male urethral opening positioned therethrough.

The sheath 1476 and fluid impermeable barrier 1402 may also include at least one vacuum relief hole 1482 that allows the chamber 1406 to remain substantially at atmospheric pressure. The vacuum relief hole 1482 may be located at any point on the sheath 1476, such as near or nearer the opening 1480. In some examples (not shown), the vacuum relief hole 1482 may extend through the cap 1484 or be disposed beneath the cap 1484. In some examples, the fluid collection assembly 1400 may not include the vacuum relief hole 1482, such as when a more complete seal as desired for the chamber 1406.

The sheath 1476 also includes at least a portion of the conduit 1412 therein, such as at least partially disposed in the chamber 1406 of the conduit 1412 only disposed in the fluid outlet 1408. For example, the conduit 1412 may extend from the sheath 1476 at the distal end 1456 to a proximal end 1458 at least proximate to the opening 1480. The proximal end 1458 may be disposed near or on the skin around the male urethral opening (e.g., on the penis or pubic area therearound). Accordingly, when a patient lays on their back, bodily fluids (e.g., urine) may aggregate near the opening 1480 against the skin of the subject. The bodily fluids may be removed from the chamber 1406 via the conduit 1412.

In some examples, the fluid impermeable barrier 1402 may be constructed of a material and/or have a thickness that allows the sheath 1476 to collapse when placed under vacuum, such as to remove air around a penis in the fluid collection assembly 1400 during use. In such examples, the conduit 1412 may extend only to or into the distal end 1456 in the chamber 1406 (e.g., not through to the area adjacent the opening).

In an example, portions of the chamber 1406 may be substantially empty due to the varying sizes and rigidity of the male penis. However, in some examples, the outermost regions of the chamber 1406 (e.g., periphery of the interior regions of the sheath 1476) may include porous material 1410 (e.g., one or more of the fluid permeable membrane 1422 and fluid permeable support 1424). For example, the porous material 1410 may be bonded to the inner surface 1418 of the fluid impermeable barrier 1402. The porous material 1410 may be positioned (e.g., at the distal end of the chamber 1406) to blunt a stream of urine from the male urethral opening thereby limiting splashing and/or to direct the bodily fluids to a selected region of the chamber 1406. Since the chamber 1406 is substantially empty (e.g., substantially all of the chamber 1406 forms a reservoir), the bodily fluids are likely to pool at a gravimetrically low point of the chamber 1406. The gravimetrically low point of the chamber 1406 may be at an intersection of the skin of an individual and the fluid collection assembly 1400, a corner formed in the sheath 1476, or another suitable location depending on the orientation of the wearer.

The porous material 1410 may include one or more of the fluid permeable membrane 1422 or the fluid permeable support 1424. The fluid permeable membrane 1422 and the fluid permeable support 1424 may be similar or identical to any of the fluid permeable membranes or fluid permeable supports as respectively disclosed herein, in one or more aspects such as material make-up or wicking ability. One or more of the fluid permeable membrane 1422 or the fluid permeable support 1424 may be disposed between the fluid impermeable barrier 1402 and a penis inserted into the chamber 1406. The fluid permeable membrane 1422 may be positioned between the fluid impermeable barrier 1402 and a penis inserted into the chamber 1406, such as between the fluid permeable support 1424 and penis of a wearer as shown. The fluid permeable support 1424 may be positioned between the fluid permeable membrane 1422 and the fluid impermeable barrier 1402. The inner surface 1418, optionally including the end of the chamber 1406 substantially opposite the opening 1404, may be covered with one or both the fluid permeable membrane 1422 or the fluid permeable support 1424. The fluid permeable support 1424 or the fluid permeable membrane 1422 may be affixed (e.g., adhered) to the fluid impermeable barrier 1402. The fluid permeable support 1424 or the fluid permeable membrane 1422 may be affixed to each other. In some examples, the porous material 1410 only includes the fluid permeable membrane 1422 or the fluid permeable support 1424.

In some examples, the fluid collection assembly 1400 includes a cap 1484 at a distal end 1456. The cap 1484 defines an interior channel through which the bodily fluids may be removed from the fluid collection assembly 1400. The interior channel is in fluid communication with the chamber 1406. The cap 1484 may be disposed over at least a portion of the distal end 1456 of one or more of the fluid impermeable barrier 1402 or the porous material 1410. The cap 1484 may be made of a polymer, rubber, or any other fluid impermeable material. The cap 1484 may be attached to one or more of the fluid impermeable barrier 1402, the porous material 1410, or the conduit 1412. The cap 1484 may have a laterally extending flange 1486 and a longitudinally extending flange 1488. The laterally extending flange 1486 may cover at least a portion of the distal end 1456 of the fluid collection assembly 1400. The longitudinally extending flange 1488 may laterally extend a distance from the sheath 1476. The longitudinally extending flange 1472 is sized and configured to receive and fluidly seal against the conduit 1412, such as within the interior channel. The conduit 1412 may extend a distance within or through the cap 1484, such as to the porous material 1410, through the porous material 1410, or to a point set-off from the porous material 1410. In the latter example, as depicted in FIG. 14B, the interior channel of the cap 1484 may define a reservoir 1416 therein.

The reservoir 1416 is an unoccupied portion of device such as in the cap 1484 and is void of other material. In some examples, the reservoir 1416 is defined at least partially by the porous material 1410 and the cap 1484. During use, the bodily fluids that are in the chamber 1406 may flow through the porous material 1410 to the reservoir 1416. The reservoir 1416 may store at least some of the bodily fluids therein and/or position the bodily fluids for removal by the conduit 1412. In some examples, at least a portion of the porous material 1410 may extend continuously between at least a portion of the opening of the interior channel and chamber 1406 to move any bodily fluid from the opening directly to the reservoir 1416.

In some examples (not shown), the fluid impermeable barrier 1402 may be disposed on or over the cap 1484, such as enclosing the cap 1484 within the chamber 1406.

In some examples, the sheath 1476 may include at least a portion of the conduit 1412 therein, such as at least partially disposed in the chamber 1406. For example, the conduit 1412 may extend from the sheath 1476 to a region at least proximate to the opening 1480. The inlet of the conduit 1412 may be positioned adjacent to the annular base 1478. The inlet of the conduit 1412 may be positioned to be adjacent or proximate to the gravimetrically low point of the chamber 1406, such as adjacent to the annular base 1478. For example, the inlet may be co-extensive with or offset from the opening 1480. In examples, the inlet may be positioned adjacent to the distal region 1444 of the sheath 1476.

The proximal end 1458 may be disposed near or on the skin around the male urethral opening (e.g., around the penis) and the inlet of the conduit 1412 may be positioned in the proximal end 1458. The outlet of the conduit 1412 may be directly or indirectly coupled to a vacuum source. Accordingly, the bodily fluids may be removed from the proximal end 1458 of the chamber 1406 via the conduit 1412.

The receptacle 1474, the sheath 1476, the cap 1484, and the conduit 1412 may be attached together using any suitable method. For example, at least two of the receptacle 1474, the sheath 1476, the cap 1484, or the conduit 1412 may be attached together using at least one of an interference fit, an adhesive, stitching, welding (e.g., ultrasonic welding), tape, any other suitable method, or combinations thereof.

In some examples (not shown), the fluid collection assembly 1400 may have a one piece design, with one or more of the sheath 1476, the receptacle 1474, and the cap 1484 being a single, integrally formed piece.

Also as shown, the conduit 1412 may be at least partially disposed with the chamber 1406 of a fluid collection assembly 1400. The conduit 1412 may extend from the distal region 1444 to the proximal end 1458. For example, the conduit 1412 may extend through the cap 1484 to a point adjacent to the receptacle 1474. The conduit 1412 is sized and positioned to be coupled to a fluid storage container or the vacuum source (FIG. 10. An outlet of the conduit 1412 may be operably coupled to the vacuum source, directly or indirectly. The inlet (e.g., open terminal end) of the conduit 1412 may be positioned within or adjacent to the chamber 1406 such as at a location expected to be at the gravimetrically low point of the fluid collection assembly 1400 during use. By positioning the inlet in a location expected to be at the gravimetrically low point of the fluid collection assembly 1400 when worn by the user, the bodily fluids introduced into the chamber 1406 may be removed via the conduit 1412 to prevent pooling or stagnation of the fluid within the chamber 1406.

The fluid collection assembly 1400 may include any of the movement enhancing features disclosed herein. In an example, the surface of the porous material 1410 that contacts the penis may include one or more grooves and/or one or more perforations formed therein. In an example, at least a portion of the fluid impermeable barrier 1402 and/or the receptacle 1474 may include a textured surface. In an example, the fluid collection assembly 1400 may be configured such that the conduit 1412 does not, when straight, extend in substantially the same direction and the longitudinal axis of the fluid collection assembly 1400. In an example, the fluid collection assembly 1400 may exhibit a plurality of different sizes.

Further examples of fluid collection assemblies that are configured to collect bodily fluids from males are disclosed in U.S. patent application Ser. No. 16/433,773 filed on Jun. 6, 2019, the disclosure of which is incorporated herein, in its entirety, by this reference.

In some examples, the vacuum source may be remotely located from the fluid collection assembly 1400. In such examples, the conduit 1412 may be fluidly connected to the fluid storage container, which may be disposed between the vacuum source and the fluid collection assembly 1400.

During operation, a male using the fluid collection assembly 1400 may discharge bodily fluids into the chamber 1406. The bodily fluids may pool or otherwise be collected in the chamber 1406. At least some of the bodily fluids may be pulled through the interior of the conduit 1412 via the inlet. The bodily fluids may be drawn out of the fluid collection assembly 1400 via the vacuum/suction provided by the vacuum source. During operation, the aperture 1462 may substantially maintain the pressure in the chamber 1406 at atmospheric pressure even though the bodily fluids are introduced into and subsequently removed from the chamber 1406.

As will be discussed in more detail below, the bodily fluids may be removed from the chamber of the fluid collection assemblies disclosed herein using a vacuum source. For example, the vacuum source may be connected to the conduit and may be configured to apply a suction force to the conduit. The suction force may pull bodily fluids that are present in the chamber into the conduit and pull the bodily fluids through the conduit and into a fluid storage container. The vacuum source may include any suitable vacuum source. However, in some embodiments, the vacuum source may have a fixed location or is otherwise not portable. In such embodiments, the vacuum source may significantly limit the mobility of the individual using the fluid collection assemblies since the fluid collection assembly is tethered to the vacuum source. As such, in some embodiments, the movement enhancing features of the fluid collection assemblies disclosed herein may include attaching the fluid collection assemblies to a portable vacuum source.

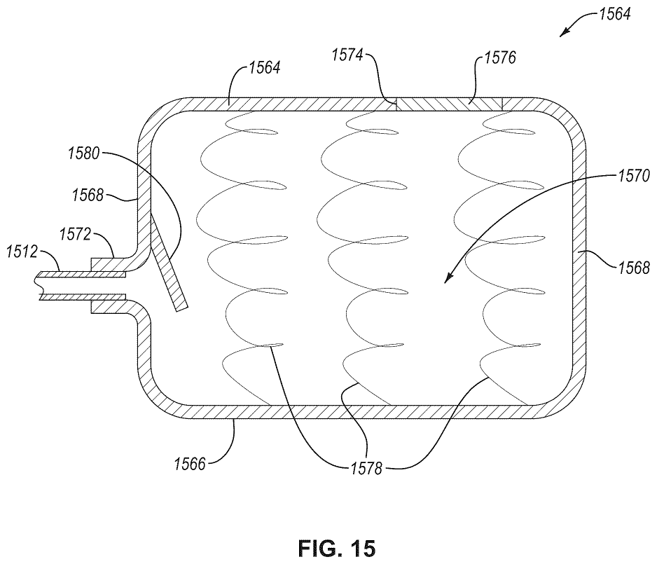

FIG. 15 is a schematic cross-sectional view of a portable vacuum source 1562, according to an embodiment. For example, the vacuum source 1562 may be used with any of the fluid collection assemblies disclosed herein. In an embodiment, the vacuum source 1562 includes at least one top wall 1564, at least one bottom wall 1566, and at least one side wall 1568 extending from the top wall 1564 to the bottom wall 1566. The top wall 1564, the bottom wall 1566, and the side wall 1568 define a chamber 1570. At least one of the top wall 1564, the bottom wall 1566, or the side wall 1568 defines a fluid inlet 1572 that is configured to be secured to the conduit 1512. Also, at least one of the top wall 1564, the bottom wall 1566, or the side wall 1568 defines a gas outlet 1574 that is configured to let gas out of the chamber 1570. The gas outlet 1574 may include a gas-permeable/water-impermeable filter 1576 that is configured to allow only gas (and not urine) to flow through the gas outlet 1574. The gas outlet 1574 and/or the filter 1576 may also include a one-way valve (not shown) that only allows the gas to flow from the chamber 1570 to an exterior of the vacuum source 1562 while preventing gas from flowing from the exterior of the vacuum source 1562 to the chamber 1570.

At least the side wall 1568 of the vacuum source 1562 is configured to be compressed (e.g., collapsed, buckled, bent, crumpled, folded, twisted, etc.) by squeezing the vacuum source 1562 with a hand. Compressing the side walls 1548 decreases the volume of the chamber 1570. Gas that is present in the chamber 1570 may escape the chamber 1570 via the gas outlet 1574 when the side wall 1548 is compressed. The vacuum source 1562 includes one or more springs 1578 that are configured expand the volume of the chamber 1570 and decompress the side wall 1548 after the vacuum source 1570 is squeezed. Expanding the volume of the chamber 1570 may apply a suction force to the conduit 1512 which, in turn, may remove bodily fluids from the fluid collection assembly (not shown). The chamber 1570 may be configured to hold the bodily fluids therein such that the vacuum source 1562 is also the fluid storage container.

The vacuum source 1562 may be configured to be squeezed multiple times until the chamber 1570 is completely filled with the bodily fluids. The vacuum source 1562 may include a one-way valve 1580 that is configured to allow bodily fluids to flow from the conduit 1512 to the chamber 1570 and restrict the bodily fluids from flowing from the chamber 1570 to the conduit 1512. As such, squeezing the vacuum source 1562 does not reintroduce the bodily fluids back into the fluid collection assembly which may cause the fluid collection assembly to leak.

FIG. 16 is a block diagram of a system 1690 for fluid collection, according to an embodiment. The system 1690 includes a fluid collection assembly 1600, a fluid storage container 1692, and a vacuum source 1664. The fluid collection assembly 1600, the fluid storage container 1692, and the vacuum source 1664 may be fluidly coupled to each other via one or more conduits 112. For example, fluid collection assembly 1600 may be operably coupled to one or more of the fluid storage container 1692 or the vacuum source 1664 via the conduit 1612. However, as previously discussed, the vacuum source 1664 and the fluid storage container 1692 may be integrally formed together. The bodily fluids collected in the fluid collection assembly 1600 may be removed from the fluid collection assembly 1600 via the conduit 1612 which protrudes into the fluid collection assembly 1600. For example, an inlet of the conduit 1612 may extend into the fluid collection assembly 1600, such as to a fluid reservoir therein. The outlet of the conduit 1612 may extend into the fluid collection assembly 1600 or the vacuum source 1664. Suction force may be introduced into the chamber of the fluid collection assembly 1600 via the inlet of the conduit 1612 responsive to suction (e.g., vacuum) force applied at the outlet of the conduit 1612.

The suction force may be applied to the outlet of the conduit 1612 by the vacuum source 1664 either directly or indirectly. The suction force may be applied indirectly via the fluid storage container 1692. For example, the outlet of the conduit 1612 may be disposed within the fluid storage container 1692 and an additional conduit 1612 may extend from the fluid storage container 1692 to the vacuum source 1664. Accordingly, the vacuum source 1664 may apply suction to the fluid collection assembly 1600 via the fluid storage container 1692. The suction force may be applied directly via the vacuum source 1664. For example, the outlet of the conduit 1612 may be disposed within the vacuum source 1664. An additional conduit 1612 may extend from the vacuum source 1664 to a point outside of the fluid collection assembly 1600, such as to the fluid storage container 1692. In such examples, the vacuum source 1664 may be disposed between the fluid collection assembly 1600 and the fluid storage container 1692.

The fluid collection assemblies 1600 may be similar or identical to any of the fluid collection assemblies disclosed herein in one or more aspects. The fluid collection assembly 1600 may be shaped and sized to be positioned adjacent to a female urethral opening or have a male urethral opening positioned therethrough (e.g., receive a penis therein). For example, the fluid collection assembly 1600 may include a fluid impermeable barrier at least partially defining a chamber (e.g., interior region) of the fluid collection assembly 1600. The fluid impermeable barrier also defines an opening extending therethrough from the external environment. The opening may be positioned adjacent to a female urethral opening or have a male urethral opening positioned therethrough. The fluid collection assembly 1600 may include a fluid permeable membrane disposed within the fluid impermeable barrier. The fluid collection assembly 1600 may include at least one porous material disposed in the chamber such as one or more of a fluid permeable support and a fluid permeable membrane.

The fluid storage container 1692 is sized and shaped to retain the bodily fluids therein. The fluid storage container 1692 may include a bag (e.g., drainage bag), a bottle or cup (e.g., collection jar), or any other enclosed container for storing bodily fluids such as urine. In some examples, the conduit 1612 may extend from the fluid collection assembly 1600 and attach to the fluid storage container 1692 at a first point therein. An additional conduit 1612 may attach to the fluid storage container 1692 at a second point thereon and may extend and attach to the vacuum source 1664. Accordingly, a vacuum (e.g., suction) may be drawn through fluid collection assembly 1600 via the fluid storage container 1692. The bodily fluids, such as urine, may be drained from the fluid collection assembly 1600 using the vacuum source 1664.

The vacuum source 1664 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1664 may provide a vacuum or suction to remove the bodily fluids from the fluid collection assembly 1600. In some examples, the vacuum source 1664 may be powered by one or more of a power cord (e.g., connected to a power socket), one or more batteries, or even manual power (e.g., a hand operated vacuum pump). In some examples, the vacuum source 1664 may be sized and shaped to fit outside of, on, or within the fluid collection assembly 1600. For example, the vacuum source 1664 may include one or more miniaturized pumps or one or more micro pumps. The vacuum sources 1664 disclosed herein may include one or more of a switch, a button, a plug, a remote, or any other device suitable to activate the vacuum source 1664.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, +2% or 0% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier defining:
at least one opening;
a chamber in fluid communication with the at least one opening; and
at least one fluid outlet;
at least one porous material disposed in the chamber, the at least one porous material including a contact surface extending across the at least one opening, the at least one porous material including one or more grooves formed in the contact surface, the one or more grooves being movement enhancing features configured to increase friction between the contact surface and a body of an individual to inhibit shifting of the fluid collection assembly when the individual moves.

3. The fluid collection assembly of claim 2, wherein the at least one porous material includes one or more thinned regions exhibiting a thickness that is less than adjacent regions of the at least one porous material, the one or more thinned regions forming the one or more grooves.

4. The fluid collection assembly of claim 2, wherein the at least one porous material includes one or more seams sewn therein, the one or more seams forming the one or more grooves.

5. The fluid collection assembly of claim 2, wherein the at least one porous material includes one or more compressed regions, the one or more compressed regions forming the one or more grooves.

6. The fluid collection assembly of claim 5, wherein the one or more compressed regions include an adhesive or melted material configured to maintain the one or more compressed regions in a compressed state.

7. The fluid collection assembly of claim 2, wherein the at least one porous material includes a plurality of ridges, the one or more grooves formed between the plurality of ridges.

8. The fluid collection assembly of claim 2, wherein the one or more grooves form concentric annular shapes.

9. The fluid collection assembly of claim 2, wherein the one or more grooves exhibit a curved shape.

10. The fluid collection assembly of claim 9, wherein the one or more grooves are generally oval.

11. The fluid collection assembly of claim 2, wherein the one or more grooves includes a plurality of non-intersecting grooves.

12. The fluid collection assembly of claim 2, wherein the one or more grooves includes a plurality of intersecting grooves.

13. The fluid collection assembly of claim 2, wherein the one or more grooves includes a plurality of grooves, each of the plurality of grooves extend for a length that is significantly greater than a width thereof.

14. The fluid collection assembly of claim 2, wherein the one or more grooves exhibit a width of about 0.1 mm to about 0.3 mm.

15. The fluid collection assembly of claim 2, wherein the one or more grooves exhibit a depth of about 0.1 mm to about 0.3 mm.

16. The fluid collection assembly of claim 2, wherein the one or more grooves are only formed on the contact surface of the at least one porous material.

17. The fluid collection assembly of claim 2, wherein the at least one porous material includes a fluid permeable top layer and a fluid permeable support, the one or more grooves extending partially into the fluid permeable top layer.

18. The fluid collection assembly of claim 2, wherein the fluid impermeable barrier exhibits a generally cylindrical shape.

19. The fluid collection assembly of claim 2, wherein the fluid impermeable barrier exhibits a generally elongated flat shape.

20. The fluid collection assembly of claim 2, further comprising a protrusion that is configured to press against and at least partially fit between labia folds of the individual, the protrusion exhibiting a width that is about 10 mm to about 50 mm.

21. The fluid collection assembly of claim 2, wherein the contact surface includes one or more perforations extending at least partially through the at least one porous material.

22. A fluid collection system, comprising:
at least one fluid collection assembly including:
a fluid impermeable barrier defining:
at least one opening;
a chamber in fluid communication with the at least one opening; and
at least one fluid outlet;
at least one porous material disposed in the chamber, the at least one porous material including a contact surface extending across the at least one opening, the at least one porous material including one or more grooves formed in the contact surface, the one or more grooves being movement enhancing features configured to increase friction between the contact surface and a body of an individual to inhibit shifting of the fluid collection assembly when the individual moves;
a fluid storage container; and
a vacuum source;
wherein the chamber of the fluid collection assembly, the fluid storage container, and the vacuum source are in fluid communication with each other that, when one or more bodily fluids are present in the chamber, a suction provided from the vacuum source to the chamber of the fluid collection assembly removes the one or more bodily fluids from the chamber and deposits the bodily fluids in the fluid storage container.
